SEQUENCE LISTING (42 sequences)

SEQ ID NO: 1 - protein, 83 aa, Artificial Sequence [sequence omitted]

SEQ ID NO: 2 - protein, 88 aa, Artificial Sequence
  PF4 Toxin Protein
IRFTDTAEQSIEDQVHHLAPFQGEQAALQSVLSLLDEIEEKISLAPKGYPVSQQASLLGVLSYRELNTGPYRVFYEFHEEQGEVAVIL

SEQ ID NO: 3 - DNA, 252 bp, Artificial Sequence
  Phd-like Antitoxin Nucleotide Sequence
atgcgagtcgagacaattagttatttgaaacgtcatgcggctgacctggatttatccgagccaatggtcgtcacgcagaacggtgttcctgcctatgtggttgagtcatatgctgagcggaagcagcgcgatgaagcaattgcgctggtgaagttgcttgcgattggctcccgccagtacgcagaaggcaagcatcgctctgttgatgatttgaaagctcgcctttccaggaggttcgctcagccagaataa

SEQ ID NO: 4 - DNA, 348 bp, Artificial Sequence
  ParE-like Toxin Nucleotide Sequence
atgtccccggtcgtcattcgttttactgataccgcagagcaaagcatcgaagaccaagtccaccacttggctccattccaaggtgaacaggctgcactccagtcagtactgagccttttggatgagattgaagagaagatttcacttgcacctaaaggttacccagtcagccagcaggcgagtcttctgggggtgctgagctatcgcgagcttaataccggcccctatcgtgttttttacgaattccacgaagagcaaggcgaggtggcagtgatcttggttttgcgacagaagcagagcgttgagcagcaattgatccgctactgcttggtggggccaatcgagtga

SEQ ID NO: 5 - protein, 115 aa, Artificial Sequence [sequence omitted]

SEQ ID NO: 6 - DNA, 264 bp, Artificial Sequence
  encodes SEQ ID NO: 2
attcgttttactgataccgcagagcaaagcatcgaagaccaagtccaccacttggctccattccaaggtgaacaggctgcactccagtcagtactgagccttttggatgagattgaagagaagatttcacttgcacctaaaggttacccagtcagccagcaggcgagtcttctgggggtgctgagctatcgcgagcttaataccggcccctatcgtgttttttacgaattccacgaagagcaaggcgaggtggcagtgatcttg

SEQ ID NO: 7 - protein, 83 aa, Pseudomonas syringae
MRVETISYLKRNAADLPLDEPLIVTQNGVPAYVVESYADRKRRDESIALVKLLAISSREYSQGKHCSADELKARLSRRFAHKE

SEQ ID NO: 8 - protein, 66 aa, Artificial Sequence
  Conserved sequence between PF4 antitoxin protein and Phd sequence of Pseudomonas syringae DC 3000
MRVETISYLKRAADLLEPVTQNGVPAYVVESYARKRDEIALVKLLAISRYGKHSDLKARLSRRFAE

SEQ ID NO: 9 - protein, 83 aa, Bacteriophage P2
VILSPAAEADLEDIADYIARRFGPSAARRYVRALETAFESLAEFPEIGRSRDEIRGGRRIVPYGSHYIFYYRVGGRVLILRVL

SEQ ID NO: 10 - DNA, 19 bp, Artificial Sequence
  Oligonucleotide Primer
agcagcgcgatgaagcaat

SEQ ID NO: 11 - DNA, 21 bp, Artificial Sequence
  Oligonucleotide Primer
tagaggccatttgtgactgga

SEQ ID NO: 12 - protein, 16 aa, Artificial Sequence
  Residues 1-15 of CoaB of Pf1 and Pf4
GVIDTSAVESAITDGC

SEQ ID NO: 13 - DNA, 27 bp, Artificial Sequence
  Repeat sequence of RF plasmid
tggagcgggcgaagggaatcgaaccct

SEQ ID NO: 14 - protein, 90 aa, Artificial Sequence
  Consensus sequence
MMYKVEIHPKALKELKKLDKKIRKKIKDKLKELLENPPPIRHGKKLRKGLSGKYRLRIGDYRLIYEIDDETLTVLVLKVGHRERIYKYAK

SEQ ID NO: 15 - protein, 84 aa, Archaeoglobus fulgidus DSM 4304
MNEVLIHKKFLDGLDSGRRSKVLDAIRMLKDFPIIRADIKKIGPKTYRLRKGEIRIIFDFDIGTNRVFVKFAASEGVFTKTEEK

SEQ ID NO: 16 - protein, 87 aa, Methanosarcina acetivorans C2A
MTYQVVLSPDFEKETKIFFKKDPVLYGRFKKTVNSILENPECGKPLRNVLKGLRRVHIGHFVLIYEIDNTNETITFLKFSPHDKAYK

SEQ ID NO: 17 - protein, 89 aa, Methanosarcina acetivorans C2A
MPYDLFILPSCKKEIDKACKNNTLLKESLSKKIQEICESPFHYKPLRNELHGMRRVHILKSFVLIFNVDENKKSVTLVSFSHYDTAYSR

SEQ ID NO: 18 - protein, 88 aa, Methanocaldococcus jannaschii
MKVLFAKTFVKDLKHVPGHIRKRIKLIIEECQNSNSLNDLKLDIKKIKGYHNYYRIRVGNYRIGIEVNGDTIIFRRVLHRKSIYDYFP

SEQ ID NO: 19 - protein, 86 aa, Ralstonia solanacearum GMI1000
MNAIHWTAWAARQLRKLDRQHQRVLVEAVGQLEAMPHCRQVRALREHRYGYRLRVGDYRVLSDWDDGIRIVDIQEVSKRDERTYRH

SEQ ID NO: 20 - protein, 89 aa, Salmonella enterica subsp. enterica serovar Typhimurium str. LT2
MTYELEFDPRALKEWHKLGDTVKAQLKKKLADVLLNPRIDSARLNGLPDCYKIKLKSSGYRLVYQVRDDVVIVFVVAVGKREHSAVYHD

SEQ ID NO: 21 - protein, 93 aa, Vibrio cholerae 01 biovar El Tor str. N16961
MKSVFVESTIFEKYRDEYLSDEEYRLFQAELMLNPKLGDVIQGTGGLRKIRVASKGKGKRGGSRIIYYFLDEKRRFYLLTIYGKNEMSDLNAN

SEQ ID NO: 22 - protein, 86 aa, Yersinia pestis CO92
MVKVDWSRKAVKQLLSIDARYRKPISEKVNKLTNFPAVDLDIKKLQMGDSQFRMRVGNYRVIFQIVEGTPVICTIQEVKRRTTATY

SEQ ID NO: 23 - protein, 102 aa, Synechocystis sp. PCC 6803
HLVNIDFTPEYRRSLKYLAKKYRNIRSDVQPIIEALQKGVISGDRLAGFGSDIYVYKLRIKNSNIQKGKSSGYRLIYLLESENSILLLTIYSKAEQEDIAAS

SEQ ID NO: 24 - protein, 98 aa, Artificial Sequence
  Consensus
MMKVILSPAAEADLEDIADYIARRFGPSAARRYVRALETAFESLAEFPEIGRSRDEIRGGRRIVPYGSHYIFYYRVGGRVLILRVLHGRRDLPRHLGD

SEQ ID NO: 25 - protein, 50 aa, Agrobacterium tumefaciens str. C58
MTGVSRHGYGTGLRSIAYRDRVIFFRVNNGELTVMRVLHGHQDISADDFK

SEQ ID NO: 26 - protein, 47 aa, Agrobacterium tumefaciens str. C58
TTYLSKKVKLTWSAFAPSDRDGIFTHIEADNPIAAIAVDDNILASVR

SEQ ID NO: 27 - protein, 97 aa, Agrobacterium tumefaciens str. C58
MPRGDKSAYTDKQKRKAEHIEEGYEDRGVSEREAERRAWATVNKESGGGKKSGSGRGHAENHASSEKGGRKGGAAAASRTKAERSASEKKGCGNTQA

SEQ ID NO: 28 - protein, 96 aa, Deinococcus radiodurans R1
MPKAWSNKDERQYEHVKDSEVKRGESPDRAEEIAARTVNKSRREEGRTPNKRTQGTGNPDAALSDLTRDELYNRAKEKGIAGRSRMSKAELVRALS

SEQ ID NO: 29 - protein, 111 aa, Pseudomonas aeruginosa PAO1 [sequence omitted]

SEQ ID NO: 30 - protein, 77 aa, Sinorhizobium meliloti 1021
MIARCAGIAAGTVPSQDCRRIISSELPEDLRFARCGQHFIVFVDNAEQVIIVDFLHARTNLPRRLAALAASKPVESH

SEQ ID NO: 31 - protein, 98 aa, Xylella fastidiosa 9a5c
MPRVIFAPEAILNIQRLRNFLHPKNTDAARRAGEAIMRGARMLGAQPHIGRPVDDMPDEYREWLIDFGDSGYVARYHIDGDTVTILAVRHHKEVGYTS

SEQ ID NO: 32 - protein, 70 aa, Xylella fastidiosa 9a5c
MRHYIATLERGIASLAEGRGAFNDMSSLFPALRMGRYEHHYVFCLPREEAPALIVAIFHERMDLMTRLAD

SEQ ID NO: 33 - protein, 97 aa, Mesorhizobium loti MAFF303099
MIPLRNGAKRRPSATYLHKHLQTLSETPALWRKLPGNLAIPADLKLDAYFSHHGRHYVFFRKLSGDRIGVISILHDRMDVPVRLAEDLQALQSRSED

SEQ ID NO: 34 - protein, 80 aa, Artificial Sequence
  Synthetic peptide
MRVETISYLKRHAADLDLSEPMVVTQNGVPAYVVESYAERKQRDEAIALVKLLAIGSRQYAEGKHRSVDDLKARLSRRFA

SEQ ID NO: 35 - protein, 68 aa, Artificial Sequence
  Conserved sequence between PF4 Antitoxin and ORF29 of Pseudomonas syringae pv syringae
MRVETISYLKRAADLLEPVVTQNGVPAYVVESYARKRDEAIALVKLLAISRYAGKHSDLKARLSRRFA

SEQ ID NO: 36 - protein, 80 aa, Pseudomonas syringae pv. syringae
MRVETISYLKRNAADLPLDEPLVVTQNGVPAYVVESYADRKRRDEAIALVKLLAISSREYAQGKHCSTDELKARLSRRFA

SEQ ID NO: 37 - protein, 77 aa, Artificial Sequence
  Synthetic peptide
RVETISYLKRHAADLDLSEPMVVTQNGVPAYVVESYAERKQRDEAIALVKLLAIGSRQYAEGKHRSVDDLKARLSRR

SEQ ID NO: 38 - protein, 35 aa, Artificial Sequence
  Comparison between SEQ ID NO: 1 (fragment) and sequence of Microbulbifer degradans 2-40
ISYLKHAALSEPVTQNGVQEALKLAGRQEGKDALR

SEQ ID NO: 39 - protein, 82 aa, Microbulbifer degradans 2-40
QIKPISYLKAHAAEVVRNLSTQVEPLVITQNGEAKAVMQGIKSYEQTQETMALLKMLALGQRQIDEGKVQPAGDVVAKLRSR

SEQ ID NO: 40 - protein, 76 aa, Artificial Sequence
  Synthetic peptide
VETISYLKRHAADLDLSEPMVVTQNGVPAYVVESYAERKQRDEAIALVKLLAIGSRQYAEGKHRSVDDLKARLSRR

SEQ ID NO: 41 - protein, 27 aa, Artificial Sequence
  Comparison between SEQ ID NO: 1 (fragment) and Geobacter metallireducens GS-15
YLKAADLPTQNGVQAILKLAGGDARLR

SEQ ID NO: 42 - protein, 81 aa, Geobacter metallireducens
IRPVTYLKSRAADLLAQVNETHRPVIITQNGEARAVLQDPESYEQMRAAIGLLKLVAQGEEDVRAGRVSEQDEIFARLERK

(19) United States
(12) Patent Application Publication
(10) Pub. No.: US 2010/0028378 A1
(43) Pub. Date: Feb. 4, 2010

(54) TOXIN-ANTITOXIN SYSTEM AND APPLICATIONS THEREOF

(75) Inventors: Mathew Thye Ngak Lau, Singapore (SG); Staffan Kjelleberg, La Perouse (AU); Jeremy Stephen Webb, Ocean Village (GB)

Correspondence Address: KNOBBE MARTENS OLSON & BEAR LLP, 2040 MAIN STREET, FOURTEENTH FLOOR, IRVINE, CA, 92614, US

(73) Assignee: Newsouth Innovations Pty. Limited, Kenisington (AU)

(21) Appl. No.: 11/719,243

(22) PCT Filed: Nov. 15, 2005
(86) PCT No.: PCT/SG2005/000389

§ 371 Date: Jun. 1, 2009

Related U.S. Application Data
(60) Provisional application No. 60/628,159, filed on Nov. 15, 2004.

(51) Int. Cl. A61K 39/00; C07K 14/00; C12N 15/11; C12N 15/00; C12N 1/21; C12P 19/34; C12P 21/00; G01N 33/566; C12Q 1/68; A61K 38/16; A61L 2/18

(52) U.S. Cl. 424/200.1; 530/324; 530/350; 536/23.1; 435/320.1; 435/252.3; 435/91.1; 435/69.1; 436/501; 435/6; 514/12; 422/28

(57) ABSTRACT

The present invention relates to the discovery of a toxin-antitoxin system in opportunistic human pathogen Pseudomonas aeruginosa and to the applications of this discovery including the stabilization of plasmids useful in the field of recombinant DNA technology for production of genes and their products. The Phd-like (prevent host death) antitoxin protein and ParE-like toxin protein of the invention are shown in FIGS. 1, 2 and 15.

All documents cited herein are incorporated by reference in their entirety.

### TECHNICAL FIELD

The present invention relates to the discovery of a toxin-antitoxin system in opportunistic human pathogen Pseudomonas aeruginosa and to the applications of this discovery including the stabilization of plasmids useful in the field of recombinant DNA technology for production of genes and their products. The invention also relates to control of bacterial growth and to the stable expression of heterologous genes.

### BACKGROUND TO THE INVENTION

Early on during the development of recombinant DNA technology, it was realized that a major challenge to that emerging technology was the stable maintenance of recombinant plasmids in bacterial cells. It was also realized that this problem stems largely from the heavy metabolic burden imposed on genetically-engineered bacterial cells as a result of high-level expression of proteins which are of no value to them.

Consequently, when propagating genetically-engineered bacterial cells that have no incentive to maintain the recombinant plasmid, over time, plasmid-free bacterial cells appear at increasing frequency. Because of the heavier metabolic burden on plasmid-harbouring bacterial cells, plasmid-free bacterial cells have higher growth rates. Accordingly, within a relatively short period of time, the bacterial culture can become dominated by plasmid-free bacterial cells, thus leading to a decreasing plasmid yield.

Various methods for ensuring the stable inheritance of plasmids have been used in the art. One common method has involved the use of antibiotics in the culture media so that those cells which carry the plasmid are selected for as the plasmids carry a gene which confers resistance to the antibiotic. However, the use of antibiotics in this manner can pose a variety of problems, in particular where the plasmids are being used in the large-scale production of heterologous proteins.

Due to the significant disadvantages associated with the use of antibiotics various alternative means for ensuring the stable maintenance of plasmids have been developed in the art. See, for example, U.S. Pat. No. 6,703,233, U.S. Pat. No. 6,413,768, U.S. Pat. No. 6,258,565, U.S. Pat. No. 4,806,471, U.S. Pat. No. 5,922,583, U.S. Pat. No. 4,760,022, and U.S. Pat. No. 6,143,518.

One method described in the above publications involves construction of a toxin-antidote killing system for plasmids expressing heterologous genes. In such a system, plasmids replicating in the cytoplasm of the bacterium express a critical antidote required by the bacterium to grow and replicate; loss of such plasmids removes the ability of the bacterium to express the antidote and results in cell death. This phenomenon of plasmid loss during bacterial replication, which results in the death of any plasmid-less bacterium, is also referred to as “post-segregational killing” or “programmed cell death” or “plasmid addiction”. Such systems avoid the use of selection markers (such as antibiotic resistance cassettes) and the need to provide external selection such as external antibiotic selection.

One example of a toxin-antidote system which has been used to enhance the maintenance of expression plasmids in bacteria is the phd-doc system which occurs naturally within the temperate bacteriophage P1, which lysogenizes Escherichia coli, as an ˜100 kb plasmid. This maintenance locus encodes two small proteins: the toxic 126 amino acid Doc protein causes death on curing of the plasmid by an unknown mechanism, and the 73 amino acid Phd antitoxin prevents host death, presumably by binding to and blocking the action of Doc.

Phd and Doc are encoded by a single transcript in which the ATG start codon of the downstream doc gene overlaps by one base the TGA stop codon of the upstream phd gene. Expression of these two proteins is therefore translationally coupled, with Phd synthesis exceeding synthesis of the toxic Doc protein.

In addition, transcription of this operon is autoregulated at the level of transcription through the binding of a Phd-Doc protein complex to a site which blocks access of RNA polymerase to the promoter of the operon as concentrations of both proteins reach a critical level. Although Doc appears to be relatively resistant to proteolytic attack, Phd is highly susceptible to cleavage. The mechanism of the plasmid-encoded phd-doc locus is therefore activated when bacteria spontaneously lose this resident plasmid, leading to degradation of the Phd antitoxin and subsequent activation of the Doc toxin which causes cell death.

Another example of a toxin-antidote system which has been used to enhance the maintenance of expression plasmids in bacteria is the par system which is described in, for example, U.S. Pat. No. 6,143,518. In the parDE system where a bacterial toxin protein, ParE, belongs to phage P2 of Escherichia coli, and has been shown to be toxic to the bacterial cell via inhibition of DNA gyrase.

Surprisingly, work by the authors on the lysogenic sequence of a novel bacteriophage termed Pf4 in Pseudomonas aeruginosa has revealed two genes each with close sequence homology to toxin-antidote genes of other Gram negative bacteria. The two genes each have conserved domain homology to toxin-antidote genes of other Gram negative bacteria. The first gene exhibits close identity with the phd (prevent host death) gene of Pseudomonas spp.; the second bears similarity in conserved domains to parE toxin gene of the E. coli parDE system. These genes are each components of different 2-component toxin-antitoxin systems, namely the phd/doc system, and the parDE of E coli. This is the first time that components of toxin-antitoxin systems from different organisms are naturally combined to form a programmed cell death operon, and increases the permutations for pairing and complementation of antitoxin-toxin systems for use in vectors.

Pseudomonas aeruginosa is an opportunistic pathogen, meaning that it exploits some break in the host defenses to initiate an infection. It causes urinary tract infections, respiratory system infections, dermatitis, conjunctivitis, otitis, soft tissue infections, bacteraemia, bone and joint infections, gastrointestinal infections and a variety of systemic infections, particularly in patients with severe burns and in cancer and AIDS patients who are immunosuppressed. Pseudomonas aeruginosa infection is a serious problem in patients hospitalized with cancer, cystic fibrosis, and burns. The case fatality rate in these patients is 50 percent.

In cystic fibrosis, progressive lung disease is the predominant cause of illness and death in people with CF. Mucus blocks the airway passages and results in a predisposition toward chronic bacterial infections. The most common bacterium to infect the CF lung is Pseudomonas aeruginosa. The lungs of most children with CF become colonized (inhabited long-term) by P. aeruginosa before their 10th birthday. The body's response to P. aeruginosa includes inflammation, which causes repeated exacerbations or episodes of intense breathing problems. Although antibiotics can decrease the frequency and duration of these attacks, the bacterium establishes a permanent residence and can never be completely eliminated from the lungs.

Pseudomonas aeruginosa is believed to reside as a biofilms in the airway mucus of cystic fibrosis patients. Biofilms are matrix-enclosed bacterial populations adherent to each other and/or to surfaces or interfaces (including automatic watering pipes, recoil hoses, water bottles, or sipper tubes), forming either single-species or mixed-species microcolonies which are phenotypically distinct from their planktonic counterparts, and which provide primitive homeostasis and metabolic cooperativity within the microcolony.

Over 99% of all bacteria live in biofilm communities. The formation of stationary, metabolically cooperative biofilms ensures protection of microcolony members from adverse environmental conditions, chemical disinfectants, and antibacterial agents. Biofilm cells have been shown to be 500 times more resistant to antibacterial agents as compared to planktonic forms.

Biofilms containing pathogenic bacteria such as Pseudomonas aeruginosa can form on a variety of devices used in biomedical research and clinical care, including endrotracheal tubes used for chronic mechanical ventilation, indwelling catheters, vascular prostheses, cardiac pacemakers, prosthetic heart valves, biliary stents, indwelling urinary catheters, chronic peritoneal dialysis catheters, extended-wear contact lenses, and artificial joints, resulting in serious infections which are unresponsive to antimicrobial therapy. Many of these same devices are used in biomedical research and clinical veterinary medical practices. Medical device manufacturers have spent decades and hundreds of millions of dollars to identify colonization-resistant materials, but have been frustrated by versatile bacteria with adaptive adhesion mechanisms.

Studies have highlighted bacteriophage genes as being among the most highly up-regulated groups of genes during biofilm development in both Gram positive and Gram negative bacteria (Stanley, N. R., R. A. Britton, A. D. Grossman, and B. A. Lazazzera. 2003. J. Bacteriol. 185:1951-1957 and, Whiteley, M., M. G. Bangera, R. E. Bumgarner, M. R. Parsek, G. M. Teitzel, S. Lory, and E. P. Greenberg. 2001. Nature 413:860-864.).

In P. aeruginosa, genes of a Pf1-like filamentous bacteriophage, which exists as a prophage within the genome of P. aeruginosa, showed up to 83.5-fold activation during biofilm development, compared with planktonic cells (Whiteley, M., et al. 2001. Nature 413:860-864). Other studies have shown that activation of Pf1 genes in biofilms is regulated by quorum-sensing in P. aeruginosa (Hentzer, M., et al. 2004. Quorum Sensing in Biofilms: Gossip in Slime City. In M. Ghannoum and G. A. O'Toole (ed.), Microbial biofilms. ASM Press, Washington, D.C. pp. 478., Wagner, V. E. et al, 2003. Journal of Bacteriology 185:2080-2095.). Moreover, activity of the Pf1-like phage is also linked to the killing and lysis of a subpopulation of P. aeruginosa cells within biofilms (Webb, J. S., et al. 2003. J. Bacteriol. 185:4585-4592.). Induction of the Pf1-like phage (here designated Pf4) in P. aeruginosa may therefore represent an important physiological and developmental event during biofilm development.

Here we show that activity of the Pf4 phage in P. aeruginosa biofilms is linked to the emergence of a subpopulation of cells with a small-colony phenotype in the effluent run-off from the biofilm. These cells exhibit high densities of filamentous phage on the cell-surface, demonstrate enhanced adhesion and microcolony development, and occur in high numbers within the biofilm run-off. Our data suggest that Pf4-SCVs play an important role in biofilm development, as well as in the colonization of new surfaces during biofilm dispersal.

### SUMMARY OF THE INVENTION

The present invention provides: (1) bacterial cells transformed with plasmids which plasmids are stably maintained without the need to provide external selection pressure; (2) methods for identifying compounds which alter the expression or activity of the proteins of the invention and which may thereby find utility in the control of P. aeruginosa growth in cystic fibrosis patients and in other P. aeruginosa diseases and in the control of P. aeruginosa biofilms; (3) bacterial cells transformed with plasmids which enable the bacterial cells to be killed as required; and (4) plasmids which enable the presence of the plasmid and containment of the cloned gene of interest in a bacterial host to be confirmed. The Phd-like (prevent host death) antitoxin protein and ParE-like toxin protein of the invention are shown in FIGS. 1 and 2. See also FIG. 15. Further aspects of the invention will also be apparent from the discussion below.

A first aspect of the invention provides a Phd-like (prevent host death) antitoxin protein which protein comprises or consists of the sequence as set forth in SEQ ID NO.1 or a functional equivalent thereof.

A second aspect of the invention provides a ParE-like toxin protein which protein comprises or consists of the sequence as set forth in SEQ ID NO.2 or 5 or a functional equivalent thereof.

A third aspect of the invention provides a nucleic acid molecule which encodes an antitoxin protein according to the first aspect of the invention.

A fourth aspect of the invention provides a nucleic acid molecule which encodes a ParE-like toxin protein according to the second aspect of the invention.

A fifth aspect of the invention provides a plasmid which comprises a nucleic acid sequence of interest and which replicates in bacteria and which is stabilized by the presence of a nucleic acid sequence according to the third aspect of the invention and a nucleic acid sequence according to the fourth aspect of the invention

A sixth aspect of the invention provides a plasmid which comprises a nucleic acid sequence of interest and which replicates in bacteria and which comprises a nucleic acid sequence encoding an antitoxin protein according to the third aspect of the invention.

A seventh aspect of the invention provides a plasmid which comprises a nucleic acid sequence of the fourth aspect of the invention (i.e. a nucleic acid sequence encoding the ParE-like toxin) whereby the expression of ParE-like toxin is driven by a constitutive or selectable expression promoter and wherein the ParE-like toxin encoding nucleic acid sequence of the fourth aspect of the invention comprises a multiple cloning site (MCS).

In one aspect of the seventh aspect of the invention a nucleic acid sequence of interest has been inserted into the MCS of the plasmid of the seventh aspect of the invention.

An eighth aspect of the invention provides a bacterium transformed with a plasmid according to the fifth, sixth or seventh aspect of the invention.

An ninth aspect of the invention provides a method to replicate DNA contained in a plasmid according to the invention which method comprises culturing bacterial cells of the eighth aspect of the invention.

A tenth aspect of the invention provides a method of producing a protein of interest, the method comprising culturing bacterial cells of the eighth aspect of the invention under conditions whereby said protein of interest is expressed from the nucleic acid sequence of interest, and recovering said protein of interest thus produced.

An eleventh aspect of the invention provides a pharmaceutical composition comprising a bacterium according to the eighth aspect of the invention.

A twelfth aspect of the invention provides a method for vaccinating a subject comprising administering to the subject an amount of a bacterial live vector vaccine sufficient to elicit an immune response wherein the bacterial live vector vaccine is a bacterium according to the eighth aspect of the invention.

A thirteenth aspect of the invention provides a bacterium according to the eighth aspect of the invention for use in medicine.

A fourteenth aspect of the invention provides the use of a bacterium according to the eighth aspect of the invention in the manufacture of a medicament for vaccinating a patient.

A fifteenth aspect of the invention provides a method for identifying an agonist or antagonist compound of a polypeptide of the first or second aspect of the invention.

In one embodiment, the method comprises contacting a test compound with a polypeptide of the first or second aspect of the invention and determining if the test compound binds to the polypeptide of the first or second aspect of the invention. The method may further comprise determining if the test compound enhances or decreases the activity of a polypeptide of the first or second aspect of the invention.

In one embodiment, the method comprises screening test compounds for their ability to agonize or antagonize the binding of the protein of the first aspect (Phd-like antitoxin) of the invention to the protein of the second aspect of the invention (the ParE-like toxin).

In a sixteenth aspect, the invention provides a method for identifying a compound that is effective to alter the expression of a target polynucleotide which encodes a polypeptide of the first or second aspect of the invention, the method comprising a) exposing a sample comprising the target polynucleotide to a test compound, and b) detecting altered expression, if any, of the target polynucleotide.

A seventeenth aspect of the invention provides a method of modulating cell (e.g. bacterial growth), the method comprising contacting the cells whose growth is to be controlled (e.g. bacteria) with a protein of the first or second aspect of the invention.

An eighteenth aspect of the invention provides a protein of the first or second aspect of the invention for use in medicine.

A nineteenth aspect there is provided the use of a protein of the first or second aspect of the invention in the manufacture of a medicament for preventing or treating an infection, e.g. a bacterial infection.

A twentieth aspect of the invention provides a pharmaceutical composition comprising a protein of the second aspect of the invention.

A twenty-first aspect of the invention provides a kit comprising a protein of the second aspect of the invention together with an agent for dismantling a biofilm.

### BRIEF DESCRIPTION OF DRAWINGS

FIG. 1. Alignment of PF4 antitoxin gene (SEQ ID NO.1) with phd sequence of Pseudomonas syringae DC 3000

FIG. 2. Alignment of PF4 toxin gene (SEQ ID NO.2) with parE sequence of bacteriophage P2 of E. coli. Identical amino acids are underlined in the Pf4 toxin sequence. Similar amino acids are underlined in the ParE toxin sequence.

FIG. 3 Wild-type colonies and SCV's derived from a) effluent run-off from a 7-day old biofilm, and b) P. aeruginosa overnight planktonic culture infected with the Pf1-like bacteriophage (SCVs only). Bar=3 mm.

FIG. 4 Transmission electron microscopy and immunogold-labelling using anti-Pf4 antibodies reveals high densities of filamentous bacteriophages on the cell-surface of SCVs. a) Wild-type P. aeruginosa cell showing a single flagellum, b) P. aeruginosa SCV7 cell with anti-Pf4 antibodies, c) Higher magnification image of Pf4 filaments tightly woven together, d) ΔpilA mutant of P. aeruginosa showing similar Pf4 filament production on the cell surface.

FIG. 5 Adhesion of wild-type and ΔpilA small colonies, SCV7, and Pf4-infected colonies to wells of tissue culture plates.

FIG. 6 Biofilms formed by SCVs show enhanced microcolony formation and large regions containing dead cells inside microcolonies. Five day-old P. aeruginosa biofilms stained with the BacLight Live/Dead stain. Biofilms were inoculated using a) wild-type b) SCV7, and c) Pf4-infected cells. Bar=50 μm.

FIG. 7 Comparison of the Pf4 genome with that of Pf1. Genes are coloured as followed: Blue, homologous genes found both on Pf1 and Pf4; green, genes occurring only on Pf4; red, new genes/ORFs identified in this study that occur only on prophage 2; grey, genes found only on Pf1 and not in Pf4. Numbers above Pf1 genes represent ORF numbers as presented in the published genome sequence of Pf1 (24). Numbers below Pf4 genes represent bp numbers within the Pf4 genome sequence.

FIG. 8 Table 1—The appearance of small colony variants (SCVs) correlates with the emergence of bacteriophage in the run-off from flow cell biofilms. Colony (CFU ml⁻¹) and bacteriophage (PFU ml⁻¹) counts in fluid run-off from flow cell biofilms.

FIG. 9 Table 2—Analysis of biofilm development in wild-type and Pf4-expressing P. aeruginosa strains using COMSTAT software. Values are means of data from 15 image stacks (5 image stacks from 3 replicate biofilms), and standard errors for each data point are shown. Values in bold are significantly higher than those of the wild-type strain by using analysis of variance (p≦0.05).

FIG. 10. DNA marker is HindIII ladder. Lane 1: Undigested pUC19; Lane 2: Digested pUC19; Lane 3: pGEM PE-like clone; Lane 4: pGEM parE-like clone 1;

FIG. 11 conserved domain comparison with relE toxin family

FIG. 12 conserved domain comparison with parE toxin family

FIGS. 13 & 14 comparisons with known and hypothesized anti-toxin of toxin-antitoxin system

FIG. 15 nucleotide sequence of the Phd-like antitoxin protein (SEQ ID NO.3) and the Par E-like toxin protein (SEQ ID NO.4), the toxin amino acid sequence encoded by SEQ ID NO. 4

FIG. 16 Graph illustrating the bacteriostatic effect of the Pf4 toxin.

FIG. 17 PCR results from Example 4.

### DETAILED DESCRIPTION OF THE INVENTION

A first aspect of the invention provides a Phd-like (prevent host death) antitoxin protein which protein comprises (and preferably consists of) the sequence as set forth in SEQ ID NO.1 or which comprises (and preferably consists of) a functional equivalent thereof.

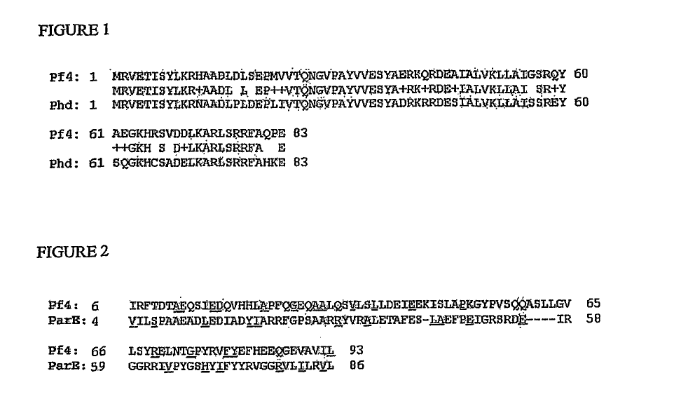

SEQ ID NO.1 refers to the Pf4 sequence of 83 amino acids set forth in FIG. 1 in the top line of the alignment.

A second aspect of the invention provides a ParE-like toxin protein which protein: (i) comprises (and preferably consists of) the sequence as set forth in SEQ ID NO.2 or SEQ ID NO.5; or (ii) which comprises (and preferably consists of) a functional equivalent of (i).

SEQ ID NO.2 refers to the Pf4 sequence of 93 amino acids set forth in the top line of the alignment in FIG. 2.

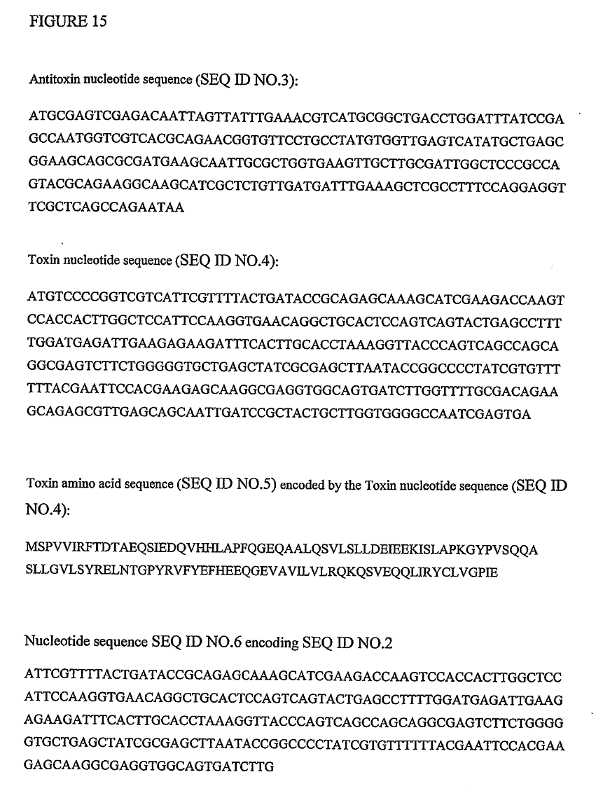

SEQ ID NO.5 refers to the Pf4 amino acid sequence in FIG. 15. As can be seen, SEQ ID No.5 comprises SEQ ID NO.2 with additional amino acid residues at the N and C termini of the SEQ ID NO:2 sequence.

The terms “protein” and “polypeptide” are used interchangeably herein.

By a “functional equivalent” of a protein as set forth for example in SEQ ID NO.1, SEQ ID NO:2 or SEQ ID NO.5 we include proteins which are homologous with the protein sequence as set forth in SEQ ID NO.1, SEQ ID NO.2 or SEQ ID NO.5 respectively, with the proviso that the homologous protein is capable of achieving stable plasmids according to the invention. Thus, for example a functional equivalent of the Phd-like antitoxin protein as set forth in SEQ ID NO.1 must be capable of achieving stable plasmids when expressed in an appropriate manner on a plasmid with the ParE-like toxin protein of SEQ ID NO.2 or SEQ ID NO.5 or when coexpressed in the same bacterium but located in separate plasmids.

Thus, in other words functional equivalents of the Phd-like antitoxin protein as set forth in SEQ ID NO:1 will retain the ability (at least qualitatively) to act as an “antidote” to the ParE-like toxin of the invention. Functional equivalents of the ParE-like toxin will retain the toxicity (at least qualitatively) of the ParE-like toxin protein of SEQ ID NO.2 or SEQ ID NO.5. In one embodiment the functional equivalents of the ParE-like toxin may also retain the ability to be “neutralised” by the Phd-like antitoxin protein as set forth in SEQ ID NO. 1. Assays for verifying the biological activity of the functional equivalents of the invention will be well within the skill of person skilled in the art. Numerous methods can be used of which some, for example, can be based on bioinformatic modelling or similar programs.

Guidance in determining which amino acid residues may be substituted, inserted, or deleted without abolishing biological or immunological activity may be found using computer programs well known in the art, for example, LASERGENE software (DNASTAR).

Moreover, persons skilled in the art will be able to readily determine experimentally if the functional equivalent is capable of achieving stable plasmids according to invention. For example, bacterial live vectors may be transformed with such expression plasmids and the rate of introduction of plasmidless cells and/or rate of growth of plasmid-containing cells can be monitored to thereby assess plasmid stability.

In the present specification, the term “stability” (and related terms) is intended to include a frequency of loss of the plasmid from the host cell of less than 2×10⁻³ per cell per generation or more preferably less than 2×10⁻⁴ per cell per generation. More preferably, the loss of the plasmid from the host cell is less than 10⁻⁵/cell/generation and yet more preferably less than 5×10⁻⁶/cell/generation. In fact, it is possible to obtain plasmids which are as stable as wild-type plasmids, i.e. with a frequency of loss of less than 3×10⁻⁶ per cell per generation. Methods for determining the rate of loss will be known to those skilled in the art. Thus, for example, rate of loss can be determined by turbidity or basic microbiological plating; or incorporation of a GFP gene and determining the intensity of expressed protein.

In a further embodiment of the invention a “functional equivalent” of a polypeptide of the first or second aspect of the invention includes a polypeptide sequence which retains an immunogenic epitope in common with a polypeptide as set forth in SEQ ID NO.1, SEQ ID NO.2 or SEQ ID NO:5. Such polypeptides may be used to invoke a useful immune response in a subject in need thereof. Such an immunogenic polypeptide may or may not retain the biological activity of the polypeptide as set forth in SEQ ID NO.1, SEQ ID NO.2 or SEQ ID NO.5.

A homologous sequence is typically at least about 80 percent sequentially identical over its entire length as compared to the reference sequence (i.e. SEQ ID NO.1, SEQ ID NO.2 or SEQ ID NO:5), typically at least about 85 percent sequentially identical, preferably at least about 90 percent sequentially identical, and most preferably about 92, 94, 95, 96, 97, 98, 99 or 99.5 percent sequentially identical, as compared to the reference sequence.

“Percent identity” refers to the percentage of sequence similarity found in a comparison of two or more amino acid sequences. Percent identity can be determined electronically, e.g., by using the MEGALIGN program (DNASTAR, Madison Wis.). This program can create alignments between two or more sequences according to different methods, e.g., the clustal method. (See, e.g., Higgins and Sharp (1988) Gene 73:237-244.) The clustal algorithm groups sequences into clusters by examining the distances between all pairs. The clusters are aligned pairwise and then in groups. The percentage similarity between two amino acid sequences, e.g., sequence A and sequence B, is calculated by dividing—the length of sequence A, minus the number of gap residues in sequence A, minus the number of gap residues in sequence B, into the sum of the residue matches between sequence A and sequence B, times one hundred. Gaps of low or of no homology between the two amino acid sequences are not included in determining percentage similarity. Alternatively, BLASTP may be used to determine percent identity.

Functional equivalents therefore include natural biological variants (for example, allelic variants or geographical variations within the species from which the polypeptides are derived) and mutants (such as mutants containing amino acid substitutions, insertions or deletions) of SEQ ID NO. 1, SEQ ID NO. 2 and SEQ ID NO. 5. Such mutants may include polypeptides in which one or more of the amino acid residues are substituted with a conserved or non-conserved amino acid residue (preferably a conserved amino acid residue) and such substituted amino acid residue may or may not be one encoded by the genetic code. Typical such substitutions are among Ala, Val, Leu and Ile; among Ser and Thr; among the acidic residues Asp and Glu; among Asn and Gln; among the basic residues Lys and Arg; or among the aromatic residues Phe and Tyr. Particularly preferred are variants in which several, i.e. between 5 and 10, 1 and 5, 1 and 3, 1 and 2 or just 1 amino acids are substituted, deleted or added in any combination. Especially preferred are silent substitutions, additions and deletions, which do not alter the properties and activities of the protein. Also especially preferred in this regard are conservative substitutions. Such mutants also include polypeptides in which one or more of the amino acid residues includes a substituent group.

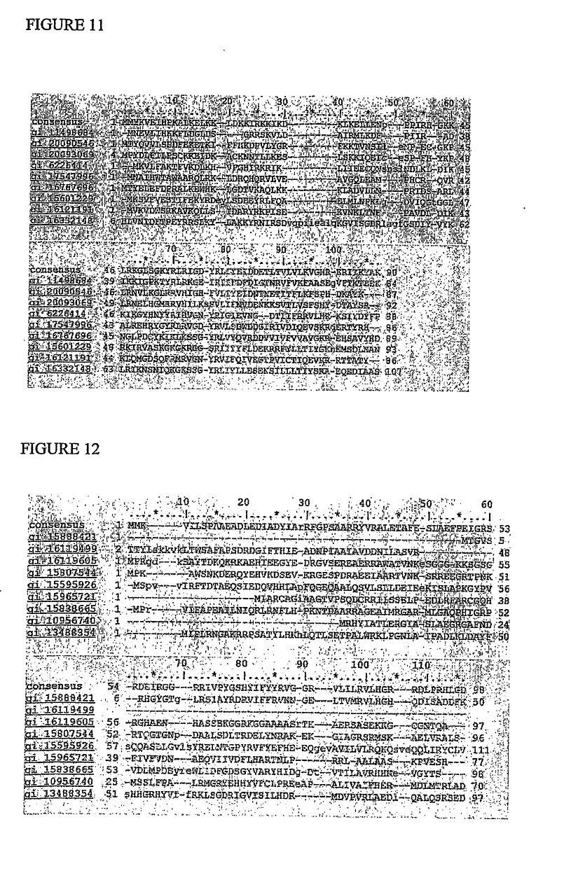

As can be seen by the conserved domain comparisons in FIGS. 11 and 12 there are a number of conserved residues in the toxin-antidote polypeptides of Gram negative bacteria toxin-antitoxin systems. As can be seen from FIG. 11, conserved residues are present at residue positions: 3, 5, 7, 9, 11, 13, 15, 16, 25, 47, 52, 69, 70, 72, 74, 77, 78, 79, 80, 81, 83, 90, 97, 98, 100 and 103 (corresponding to residues Y, V, I, P, A, K, L, K, R, N, P, Y, R, R, G, Y, R, L, I, Y, I, V, H, R, E and Y of the consensus sequence). As can be seen from FIG. 11, similar residues are present at residue positions: 4, 6, 8, 10, 12, 14, 16, 23, 24, 26, 27, 28, 29, 40, 41, 42, 43, 44, 45, 46, 53, 55, 59, 60, 61, 62, 63, 64, 65, 66, 67, 68, 71, 73, 75, 82, 84, 85, 88, 89, 91, 92, 93, 94, 95, 96, 101, 102 and 104 (corresponding to residues K, E, H, K, L, E, K, K, I, K, K, I, K, K, L, K, E, L, L, E, P, R, K, K, L, R, K, G, L, S, G, K, L, F, D, E, D, D, L, T, L, V, L, K, V, G, R, I and K of the consensus sequence). The skilled person will also be able to determine the conserved and similar amino acid residues in the sequences shown in FIG. 12. See also FIGS. 1 and 2 which indicate the conserved and similar residues of SEQ ID Nos. 1 and 2 with the Phd and ParE amino acid sequences respectively.

It will be appreciated by those skilled in the art that it may desirable for the amino acid residues of SEQ ID NOs 1, 2 and 5 present at the conserved positions (and optionally also at the similar residue positions) to be retained (or if replaced, to be replaced with conservative amino acid substitutions).

Examples of functional equivalents include fragments of the aforementioned polypeptides in which one or more amino acids (e.g. at least 1, 2, 3, 4, 5, 10, 15, 20, 25, 30, 35, 45 or 50 amino acids) have been deleted. The fragments of the invention retain the ability (at least qualitatively) to act as an “antidote” to the ParE-like toxin of the invention or retain the toxicity (at least qualitatively) of the ParE-like toxin protein. The fragments may be more, the same or less potent as antidotes/toxins as the polypeptides set forth in SEQ ID NO. 1 and SEQ ID Nos 2 or 5 respectively.

Alternatively or additionally, the fragments retain an immunogenic epitope in common with a polypeptide as set forth in SEQ ID NO.1, SEQ ID NO.2 or SEQ ID NO.5.

The fragments of the invention may contain single or multiple amino acid deletions from either (and optionally from both) terminus of the protein and/or from internal stretches of the primary amino acid sequence.

The fragments should comprise at least n consecutive amino acids from the sequence (e.g. SEQ ID NO. 1, 2, or 5 or a functional equivalent of SEQ ID NO. 1, 2 or 5) wherein n preferably is 7 or more (for example, 10, 15, 20, 30, 40, 50, 60, 70 or 80 or more).

The fragments of the invention may be “free-standing”, i.e. not part of or fused to other amino acids or polypeptides, or they may be comprised within a larger polypeptide of which they form a part or region. When comprised within a larger polypeptide, the fragment of the invention most preferably forms a single continuous region. Additionally, several fragments may be comprised within a single larger polypeptide.

In one embodiment the protein of the first or second aspect of the invention consists of the sequence as set forth in SEQ ID NO.1 or SEQ ID NO.2 or 5 respectively or a fragment thereof.

In another embodiment the protein of the first or second aspect of the invention consists of a functional equivalent of SEQ ID NO.1 or SEQ ID NO.2 or 5 or a fragment thereof.

In one embodiment of the invention, the protein of the first or second aspect of the invention may comprise additional sequences with the proviso that the said additional sequence(s) do/does not adversely interfere with the function of the protein. Thus, in one example the protein of the first or second aspect of the invention may be provided in the form of a fusion protein. In one embodiment, a polypeptide of the first or second aspect of the invention may comprise additional sequence(s) which increase the antigenicity of the polypeptide. For instance, it may be conjugated to a bacterial toxoid, such as a toxoid from diphtheria, tetanus, cholera, H. pylori, and other pathogens.

Thus, the polypeptides of the first aspect of the invention include: (a) polypeptides comprising or consisting of SEQ ID No. 1, 2 or 5; (b) functional equivalents of (a); (c) fragments of (a) and (b); and fusion proteins comprising (a), (b) or (c).

The polypeptides and nucleic acid molecules of the present invention are “isolated”. The term isolated as used herein means altered “by the hand of man” from its natural state; i.e., if it occurs in nature, it has been changed or removed from its original environment, or both. For example, a naturally occurring polypeptide naturally present in a bacterium is not “isolated”, but the same polypeptide separated from the coexisting materials of its natural state is “isolated”, as the term is employed herein.

In addition to the utilities outlined below, a polypeptide of the first or second aspect of the invention may find utility as a biocontrol agent. A nucleic acid sequence encoding a polypeptide of the second aspect (toxin) of the invention may be located in the cell's chromosome and a nucleic acid sequence encoding a polypeptide of the first aspect of the invention (antitoxin) may be located in the plasmid. Cells comprising a nucleic acid sequence encoding a polypeptide of the second aspect of the invention in its chromosome and a plasmid comprising a nucleic acid sequence encoding a polypeptide of the first aspect of the invention also form an aspect of the invention. The nucleic acid sequence encoding a polypeptide of the first or second aspect of the invention (toxin) may be under the control of a constitutive or inducible promoter. In this way the nucleic acids of the invention may be used to engineer cells such as bacteria which express (either naturally or by virtue of genetic engineering) genes of interest such as genes which express enzymes which may be useful for mineral extraction or in bioremediation in such a manner which would reduce concerns of releasing genetically engineered organisms into the environment as if such cells lose the plasmid on which the antitoxin is expressed then the cells will then die thereby limiting their spread into the environment.

A polypeptide of the first or second aspect of the invention may also find utility as an immunogen to invoke an immune response against a bacteriophage such as a pf4 bacteriophage. Where a polypeptide of the first or second aspect of the invention is used an immunogen it may be presented to the patient in combination with an adjuvant and/or conjugated with one or more additional sequences which increase the antigencity of the polypeptide of the first or second aspect of the invention.

The use (either in vivo or in vitro) of a protein according to the first or second aspect of the invention as an antitoxin or toxin respectively is provided by the present invention.

The use (either in vivo or in vitro) of a protein according to the first or second aspect of the invention in a method of biocontrol is also provided by the present invention.

Also provided is the prophylactic or therapeutic use of a protein according to the first or second aspect of the invention as an immunogen to induce an immune response against a polypeptide of the first or second aspect of the invention. Such an immune response may be useful in patients in need of protection against bacteria which are infected with the pf4 bacteriophage and other bacteria which express proteins of the first or second aspect of the invention by virtue of being infected with other bacteriophages. Thus, protection against P. syringae and other bacteria may be provided.

A third aspect of the invention provides a nucleic acid molecule which encodes an antitoxin protein according to the first aspect of the invention.

A fourth aspect of the invention provides a nucleic acid molecule which encodes a toxin protein according to the second aspect of the invention.

It will be appreciated by those skilled in the art that as a result of the degeneracy of the genetic code, a multitude of nucleic acid molecules encoding the proteins of the first and second aspect of the invention, some bearing minimal homology to the polynucleotide sequences of any known and naturally occurring gene, may be produced. Thus, the invention contemplates each and every possible variation of polynucleotide sequence that could be made by selecting combinations based on possible codon choices.

Nucleic acid molecules of the present invention may be in the form of RNA, such as mRNA, or in the form of DNA, including, for instance cDNA, synthetic DNA or genomic DNA. Such nucleic acid molecules may be obtained by cloning, by chemical synthetic techniques or by a combination thereof. The nucleic acid molecules can be prepared, for example, by chemical synthesis using techniques such as solid phase phosphoramidite chemical synthesis, from genomic or cDNA libraries or by separation from an organism. RNA molecules may generally be generated by the in vitro or in vivo transcription of DNA sequences.

The nucleic acid molecules may be double-stranded or single-stranded. Single-stranded DNA may be the coding strand, also known as the sense strand, or it may be the non-coding strand, also referred to as the anti-sense strand.

The term “nucleic acid molecule” also includes analogues of DNA and RNA, such as those containing modified backbones.

In one embodiment of the fourth aspect of the invention there is provided a nucleic acid molecule which comprises or consists of a nucleic acid sequence as set forth in SEQ ID NO 3, 4 or 6 or a degenerate version thereof:

Antitoxin:

(SEQ ID NO 3)

ATGCGAGTCGAGACAATTAGTTATTTGAAACGTCATGCGGCTGACCTGGA
TTTATCCGAGCCAATGGTCGTCACGCAGAACGGTGTTCCTGCCTATGTGG
TTGAGTCATATGCTGAGCGGAAGCAGCGCGATGAAGCAATTGCGCTGGTG
AAGTTGCTTGCGATTGGCTCCCGCCAGTACGCAGAAGGCAAGCATCGCTC
TGTTGATGATTTGAAAGCTCGCCTTTCCAGGAGGTTCGCTCAGCCAGAAT
AA
Toxin

(SEQ ID NO 4)

ATGTCCCCGGTCGTCATTCGTTTTACTGATACCGCAGAGCAAAGCATCGA
AGACCAAGTCCACCACTTGGCTCCATTCCAAGGTGAACAGGCTGCACTCC
AGTCAGTACTGAGCCTTTTGGATGAGATTGAAGAGAAGATTTCACTTGCA
CCTAAAGGTTACCCAGTCAGCCAGCAGGCGAGTCTTCTGGGGGTGCTGAG
CTATCGCGAGCTTAATACCGGCCCCTATCGTGTTTTTTACGAATTCCACG
AAGAGCAAGGCGAGGTGGCAGTGATCTTGGTTTTGCGACAGAAGCAGAGC
GTTGAGCAGCAATTGATCCGCTACTGCTTGGTGGGGCCAATCGAGTGA
Nucleotide sequence
SEQ ID NO. 6 encoding SEQ ID NO. 2
ATTCGTTTTACTGATACCGCAGAGCAAAGCATCGAAGACCAAGTCCACCA
CTTGGCTCCATTCCAAGGTGAACAGGCTGCACTCCAGTCAGTACTGAGCC
TTTTGGATGAGATTGAAGAGAAGATTTCACTTGCACCTAAAGGTTACCCA
GTCAGCCAGCAGGCGAGTCTTCTGGGGGTGCTGAGCTATCGCGAGCTTAA
TACCGGCCCCTATCGTGTTTTTTACGAATTCCACGAAGAGCAAGGCGAGG
TGGCAGTGATCTTG

Typically, the nucleic acid molecules of the fourth aspect of the invention include variant and fragment sequences, wherein said variants or fragments encode a polypeptide which retains immunological (i.e. it retains an immunological epitope) or biological activity, i.e. the ability to achieve stable plasmids according to the invention or to exhibit toxic or antidote properties as discussed in relation to the first and second aspects of the invention. Such fragments and variants can be located and isolated using standard techniques in molecular biology, without undue trial and experimentation. By “retains biological activity” we include where biological activity is retained to at least some degree, i.e. the biological activity may or may not be quantitatively retained. By “retains immunological activity” we include where immunological activity is retained to at least some degree, i.e. the immunological activity may or may not be quantitatively retained.

The term “fragment” as used herein includes a reference to a nucleic acid or polypeptide molecule that encodes a constituent or is a constituent of a particular polypeptide/nucleic acid or variant functional equivalent thereof. In terms of the polypeptide the fragment possesses qualitative biological activity in common with the polypeptide in question. A fragment of a nucleic acid sequence encodes a polypeptide which retains qualitative immunological or biological activity of the polypeptide. The fragment may be physically derived from the full-length polypeptide/nucleic acid or alternatively may be synthesised by some other means, for example chemical synthesis.

The term “variant” as used herein includes a reference to substantially similar sequences. Generally, nucleic acid sequence variants of the invention encode a polypeptide which retains qualitative biological activity or an immunogenic epitope in common with the polypeptide encoded by the “non-variant” nucleic acid sequence. Variant nucleic acid sequences include nucleic acid sequences which exhibit homology with the corresponding reference sequence (e.g. SEQ ID NO. 3, 4 or 6 or a degenerate version thereof).

A homologous sequence is typically at least about 70 percent sequentially identical as compared to the reference sequence, typically at least about 85 percent sequentially identical, preferably at least about 90 or 95 percent sequentially identical, and most preferably about 96, 97, 98 or 99 percent sequentially identical, as compared to the reference sequence.

In one embodiment of the invention, the nucleic acid molecule comprises a sequence having a sequence identity of at least 70%, 80%, 85%, 90%, 92%, 95%, 97%, 98%, or 99% to a nucleotide sequence according to SEQ ID NO:3, 4 or 6 or to sequences corresponding thereto within the degeneration of the genetic code.

“Variations” of the gene also include genes in which one or more relatively short stretches (for example 20 to 50 nucleotides) have a high degree of homology (at least 40% or 50% and preferably at least 70, 80, 85%, 90 or 95%) with equivalent stretches of a nucleic acid sequence of the invention (e.g. SEQ ID NO:3, 4 or 6 or sequences corresponding thereto within the degeneration of the genetic code) even though the overall homology between the two nucleic acid sequences may be much less. This is because important active or binding sites may be shared even when the general architecture of the encoded protein is different. In this regard, it is noted that the two genes of the invention (encoding the toxin and antitoxin) each have conserved domain homology to toxin-antidote genes of other Gram negative bacteria. “Variants” of the sequences of the invention may exhibit a high degree of homology to such conserved domains.

The degree of sequence identity between two nucleic acid sequences may be determined by means of computer programs known in the art such as GAP provided in the GCG program package (Program Manual for the Wisconsin Package, Version 8, August 1996, Genetics Computer Group, 575 Science Drive, Madison, Wis., USA 53711) (Needleman, S. B. and Wunsch, C.D., (1970), Journal of Molecular Biology, 48, 443-453). Using GAP with the following settings for DNA sequence comparison: GAP creation penalty of 5.0 and GAP extension penalty of 0.3.

Nucleic acid molecules may be aligned to each other using the Pileup alignment software, available as part of the GCG program package, using, for instance, the default settings of gap creation penalty of 5 and gap width penalty of 0.3.

The nucleic acid molecule may also include within its scope a variant capable of hybridising to the nucleic acid molecules of the invention, for instance the nucleic acid sequences defined in SEQ ID NOS: 3, 4 or 5 under conditions of low stringency, more preferably, medium stringency and still more preferably, high stringency. Low stringency hybridisation conditions may correspond to hybridisation performed at 50° C. in 2×SSC.

Suitable experimental conditions for determining whether a given nucleic acid molecule hybridises to a specified nucleic acid may involve presoaking of a filter containing a relevant sample of the nucleic acid to be examined in 5×SSC for 10 min, and prehybridization of the filter in a solution of 5×SSC, 5×Denhardt's solution, 0.5% SDS and 100 μg/ml of denatured sonicated salmon sperm DNA, followed by hybridisation in the same solution containing a concentration of 10 ng/ml of a ³²P-dCTP-labeled probe for 12 hours at approximately 45° C., in accordance with the hybridisation methods as described in Sambrook et al. (1989; Molecular Cloning, A Laboratory Manual, 2nd edition, Cold Spring Harbour, N.Y.).

The filter is then washed twice for 30 minutes in 2×SSC, 0.5% SDS at least 55° C. (low stringency), at least 60° C. (medium stringency), at least 65° C. (medium/high stringency), at least 70° C. (high stringency), or at least 75° C. (very high stringency). Hybridisation may be detected by exposure of the filter to an x-ray film.

Further, there are numerous conditions and factors, well known to those skilled in the art, which may be employed to alter the stringency of hybridisation. For instance, the length and nature (DNA, RNA, base composition) of the nucleic acid to be hybridised to a specified nucleic acid; concentration of salts and other components, such as the presence or absence of formamide, dextran sulfate, polyethylene glycol etc; and altering the temperature of the hybridisation and/or washing steps.

Further, it is also possible to theoretically predict whether or not two given nucleic acid sequences will hybridise under certain specified conditions. Accordingly, as an alternative to the empirical method described above, the determination as to whether a variant nucleic acid sequence will hybridise to the nucleic acid molecule defined in accordance with the fourth aspect of the invention (e.g. the nucleic acid of SEQ ID NO: 3, 4 or 6), can be based on a theoretical calculation of the Tₘ (melting temperature) at which two heterologous nucleic acid sequences with known sequences will hybridise under specified conditions, such as salt concentration and temperature.

In determining the melting temperature for heterologous nucleic acid sequences (Tₘ₍ₕₑₜₑᵣₒ₎) it is necessary first to determine the melting temperature (Tₘ₍ₕₒₘₒ₎) for homologous nucleic acid sequence. The melting temperature (Tₘ₍ₕₒₘₒ₎) between two fully complementary nucleic acid strands (homoduplex formation) may be determined in accordance with the following formula, as outlined in Current Protocols in Molecular Biology, John Wiley and Sons, 1995, as:

Tₘ₍ₕₒₘₒ₎=81.5° C.+16.6(log M)+0.41(% GC)−0.61 (% form)−500/L

M=denotes the molarity of monovalent cations,

% GC % guanine (G) and cytosine (C) of total number of bases in the sequence,

% form=% formamide in the hybridisation buffer, and

L=the length of the nucleic acid sequence.

Tₘ determined by the above formula is the Tₘ of a homoduplex formation (Tₘ₍ₕₒₘₒ₎) between two fully complementary nucleic acid sequences. In order to adapt the Tₘ value to that of two heterologous nucleic acid sequences, it is assumed that a 1% difference in nucleotide sequence between two heterologous sequences equals a 1° C. decrease in Tₘ. Therefore, the Tₘ(hetero) for the heteroduplex formation is obtained through subtracting the homology % difference between the analogous sequence in question and the nucleotide probe described above from the Tₘ₍ₕₒₘₒ₎.

A fifth aspect of the invention provides a plasmid which comprises a nucleic acid sequence of interest and which replicates in bacteria (by which we mean that the plasmid is capable of replicating in bacteria) and which is stabilized by the presence of a nucleic acid sequence according to the third aspect of the invention and a nucleic acid sequence according to the fourth aspect of the invention.

A sixth aspect of the invention provides a plasmid which comprises a nucleic acid sequence of interest and which replicates in bacteria and which comprises a nucleic acid sequence encoding an antitoxin protein according to the third aspect of the invention.

The plasmids of the sixth aspect of the invention can be used to transform host cells in which the host chromosome is irreversibly altered so as to produce a protein according to the second aspect of the invention which is toxic to the bacterium. Expression of the protein of the second aspect of the invention may be under the control of a selectable or constitutive promoter. When the host cell expresses the protein of the second aspect of the invention, the plasmid will be stabilised as loss of the plasmid will result in the loss of the antitoxin from the cell and the toxin encoded by the bacterial chromosome will no longer be neutralised thereby leading to cell death.

Suitably, the plasmids of the sixth aspect of the invention lack functional genetic material that encodes a toxin protein of the second aspect of the invention.

A seventh aspect of the invention provides a plasmid which comprises a nucleic acid sequence of the fourth aspect of the invention (i.e. a nucleic acid sequence encoding the ParE-like toxin protein) whereby the expression of the ParE-like toxin protein is driven by a promoter (e.g. a constitutive or selectable expression promoter) and wherein the ParE-like toxin protein encoding the nucleic acid sequence of the fourth aspect of the invention comprises a cloning site e.g. a multiple cloning site (MCS) to thereby facilitate the insertion of a nucleic acid sequence of interest. Hence, the seventh aspect of the invention provides for the use of nucleic acid sequence of the fourth aspect of the invention as a target for insertional inactivation. A nucleic acid sequence of interest may be inserted into the multiple cloning site/multiple cloning site. Disruption of the parE-like gene would follow so that functional ParE-like toxin protein would not be expressed from cells harbouring a plasmid comprising the nucleic acid sequence of interest. Thus, in one aspect of the seventh aspect of the invention a nucleic acid sequence of interest has been inserted into the multiple cloning site/MCS of the plasmid of the seventh aspect of the invention.

Suitably, the plasmids of the seventh aspect of the invention comprise a selectable marker such as a gene which encodes protein(s) which confers resistance to an antibiotic. By the inclusion of such a selectable marker those cells which comprise the plasmid of the seventh aspect of the invention may be selected for. Hence, the plasmids of the seventh aspect of the invention provide a means to ensure: (i) the plasmid comprises the nucleic acid sequence of interest; and (ii) the cells comprise the plasmid.

The plasmids of the fifth, sixth and seventh aspects of the invention include genetic material which upon transformation into a suitable host is: (i) capable of effecting production or expression of the nucleic acid sequence of interest; and (ii) capable of effecting expression of the toxin protein of the second aspect of the invention (although of course this is subject to the proviso that in the plasmids of the seventh aspect of the invention the parE-like gene may be disrupted and hence there may be no functional ParE-like expression). The plasmids of the fifth aspect of the invention additionally include genetic material which effects expression of the antitoxin protein of the first aspect of the invention. It will be appreciated that the plasmids of the fifth and sixth may be maintained in bacterial cells without any external selection pressure.

Appropriate regulatory sequences and the like for ensuring production/expression of the nucleic acid sequence of interest, the toxin protein of the invention and the antitoxin protein of the invention will be well known to those skilled in the art. Thus, features which may be included in the plasmids include promoters, further regulatory and/or enhancer functions, for example transcriptional or translational control sequences such as start or stop codons, transcriptional initiators or terminators, ribosomal binding sites etc.

In the case of the plasmids of the fifth aspect of the invention, in one embodiment the toxin and antitoxin proteins are co-expressed under the control of a single promoter. In an alternative embodiment the two proteins are expressed separately and, under the control of different promoters.

The promoters used in the construction of the plasmids of the fifth and sixth aspects of the invention may be constitutive promoters or inducible promoters. Preferably, the toxin and antitoxin sequences are under the control of inducible promoters. By using inducible promoters, death of the host cells can be controlled by manipulating culture conditions. Different inducible promoters can be used for the antitoxin and toxin sequence. In this way, cells can be selectively killed off when required. In addition, via replica plating on media with respective inducers of each of the genes, presence of the plasmid and containment of the cloned gene of interest can be confirmed.

To express the nucleic acid sequences of interest, the plasmids of the invention conveniently contain one or more sites for insertion of a cloned gene, e.g. one or more restriction sites, located downstream of the promoter region. Preferably, multiple, e.g. at least 2 or 3, up to 20 or more, such insertion sites are contained. Vectors containing multiple restriction sites have been constructed, containing e.g. 20 unique sites in a polylinker. Suitable cloning sites for insertion of a desired gene are well known in the art and widely described in the literature, as are techniques for their construction and/or introduction into the vectors of the invention (see e.g. Sambrook et al.).

Persons skilled in the art will appreciate that a wide variety of nucleic acid sequences may be desirable and thus many different types of nucleic acid sequences of interest will exist.

By a nucleic acid sequence of interest we include nucleic acid sequences which sequences are themselves of interest (e.g. sequences which act as sRNAi's or antisense nucleic acids etc.) and also nucleic acid sequences which encode polypeptides of interest. The polypeptides of interest may be polypeptides which are in themselves useful (e.g. therapeutically useful proteins) or which may be useful in the production or degradation of a desired or undesired product respectively.

The nucleic acid sequence of interest which is present in the plasmids according to the invention can be any sequence which encodes a protein which is of pharmaceutical or agrifood interest or which can be used for biocatalysis. The sequence can be a structural gene, a complementary DNA sequence, a synthetic or semi-synthetic sequence, etc.

The nucleic acid sequence of interest which is present in the plasmids according to the invention can be a nucleic acid sequence which encodes one or more enzymes. The enzyme(s) may, for example, be is useful in bioremediation or in mining, e.g. in mineral extraction such as gold extraction.

Preferably, the nucleic acid sequence encodes a protein of pharmaceutical interest which is selected, for example, from among enzymes, blood products, hormones, lymphokines (interleukins, interferons, TNF, etc.), growth factors, neurotransmitters or their precursors or enzymes for synthesizing them.

Generally, the nucleic acid sequence of interest will be a gene which is not naturally related to the plasmid.

Whilst it is generally envisaged that the desired product is a protein, in some embodiments it may be that nucleic acid is the desired product. This may be the case where nucleic acid is desired for DNA immunization or gene therapy. See U.S. Pat. No. 5,922,583 in this regard.

An eighth aspect of the invention provides a bacterium transformed with a plasmid according to the fifth, sixth or seventh aspect of the invention.

Methods for introducing expression vectors into host cells and in particular methods of transformation of bacteria are well known in the art and widely described in the literature, including for example in Sambrook et al., (supra). Electroporation techniques are also well known and widely described.

The range of possible host cells is broad and includes Gram-negative bacteria, as well as Gram-positive bacteria. Suitable Gram-negative bacteria include all enteric species, including, for example, Escherichia sp., Salmonella, Klebsiella, Proteus and Yersinia. and non-enteric bacteria including Azotobacter sp., Pseudomonas sp., Xanthomonas sp., Caulobacter sp, Acinetobacter sp., Aeromonas sp., Agrobacterium sp., Alcaligenes sp., Bordatella sp., Haemophilus Influenzae, Methylophilus methylotrophus, Rhizobium sp. and Thiobacillus sp. Gram-positive bacterial hosts which may be used include Clavibacter sp.

In one embodiment, the host cell is P. aeruginosa.

In another embodiment the host cell is E. coli.

A ninth aspect of the invention provides a method to replicate DNA contained in a plasmid of the invention which method comprises culturing bacterial cells of the eighth aspect of the invention.

Where the bacterial cells comprise a plasmid according to the fifth or sixth aspect of the invention, a method for the stable replication of DNA is provided as the viability of said cells is dependent on the presence of said plasmid in said cells.

Where plasmid DNA is the desired product, the method of the ninth aspect of the invention may further comprise recovering the desired DNA from the culture.

A tenth aspect of the invention provides a method of producing a protein of interest, the method comprising culturing bacterial cells of the eighth aspect of the invention under conditions whereby said protein of interest is expressed from the nucleic acid sequence of interest, and recovering said protein of interest thus produced.

Where the bacterial cells comprise a plasmid according to the fifth or sixth aspect of the invention, the cells are suitably cultured under conditions wherein the viability of said cells is dependent on the presence of said plasmid in said cells. Thus, where the toxin and/or antitoxin gene are under the control of inducible promoters appropriate culture conditions are provided to activate of the promoters. In this manner the plasmid is stabilised.

Preferably, the bacterial cultivation proceeds for at least 100 generations of the bacteria.

Whilst the present invention avoids the need for external selection pressure (e.g. antibiotics) the plasmids of the present invention may nevertheless include one or more genes conferring antibiotic resistance or other selectable markers. This may be useful in ensuring the correct plasmid has been taken up by the host cell.

Once the protein of interest has been recovered from the culture it may be processed as desired.

In one embodiment the recovered protein it may be cleaved if the protein is expressed in the form of a fusion protein or as a pre-, pro- or prepro-protein that can be activated by cleavage of the pre-, pro- or prepro-portion to produce an active mature polypeptide. In such polypeptides, the pre-, pro- or prepro-sequence may be a leader or secretory sequence or may be a sequence that is employed for purification of the mature polypeptide sequence.

In another embodiment, the recovered protein may be purified.

An eleventh aspect of the invention provides a pharmaceutical composition comprising a bacterium according to the eighth aspect of the invention. Such bacteria may be used to deliver antigens to a host immune system by expressing the antigens from genetic material contained within a bacterial live vector. The antigens may include a wide variety of proteins and/or peptides of bacterial, viral, parasitic or other origin. In another aspect, the antigens encoded by the expression plasmids of the present invention are cancer vaccines. In yet another aspect, the antigens encoded by these plasmids are designed to provoke an immune response to autoantigens, B cell receptors and/or T cell receptors which are implicated in autoimmune or immunological diseases. For example, where inappropriate immune responses are raised against body tissues or environmental antigens, the vaccines of the present invention may immunize against the autoantigens, B cell receptors and/or T cell receptors to modulate the responses and ameliorate the diseases. For example, such techniques can be efficacious in treating myasthenia gravis, lupus erythematosis.

Among the bacterial live vectors currently under investigation are attenuated enteric pathogens (e.g., Salmonella typhi, Shigella, Vibrio cholerae), commensals (e.g., Lactobacillus, Streptococcus gordonii) and licensed vaccine strains (e.g., BCG). S. typhi is a particularly attractive strain for human vaccination.

Where the transformed bacterial cells are administered to a subject, they are administered in an amount necessary to elicit an immune response which confers immunity to the subject for the protein or peptide. The subject is preferably a mammal (e.g. a human), but may also be another animal, such as a dog, horse, or chicken.

It is contemplated that the bacterial live vector vaccines of the present invention will be administered in pharmaceutical formulations for use in vaccination of individuals, preferably humans. Such pharmaceutical formulations may include pharmaceutically effective carriers, and optionally, may include other therapeutic ingredients, such as various adjuvants known in the art.

The carrier or carriers must be pharmaceutically acceptable in the sense that they are compatible with the therapeutic ingredients and are not unduly deleterious to the recipient thereof. The therapeutic ingredient or ingredients are provided in an amount and frequency necessary to achieve the desired immunological effect.

The mode of administration and dosage forms will affect the therapeutic amounts of the compounds which are desirable and efficacious for the vaccination application. The bacterial live vector materials are delivered in an amount capable of eliciting an immune reaction in which it is effective to increase the patient's immune response to the expressed mutant holotoxin or to other desired heterologous antigen(s). An immunizationally effective amount is an amount which confers an increased ability to prevent, delay or reduce the severity of the onset of a disease, as compared to such abilities in the absence of such immunization. It will be readily apparent to one of skill in the art that this amount will vary based on factors such as the weight and health of the recipient, the type of protein or peptide being expressed, the type of infecting organism being combated, and the mode of administration of the compositions.

The modes of administration may comprise the use of any suitable means and/or methods for delivering the bacterial live vector vaccines to a corporeal locus of the host animal where the bacterial live vector vaccines are immunostimulatively effective.

Delivery modes may include, without limitation, parenteral administration methods, such as subcutaneous (SC) injection, intravenous (IV) injection, transdermal, intramuscular (IM), intradermal (ID), as well as non-parenteral, e.g., oral, nasal, intravaginal, pulmonary, ophthalmic and/or rectal administration.

The dose rate and suitable dosage forms for the bacterial live vector vaccine compositions of the present invention may be readily determined by those of ordinary skill in the art without undue experimentation, by use of conventional antibody titer determination techniques and conventional bioefficacy/biocompatibility protocols. Among other things, the dose rate and suitable dosage forms depend on the particular antigen employed, the desired therapeutic effect, and the desired time span of bioactivity.

Formulations of the present invention can be presented, for example, as discrete units such as capsules, cachets, tablets or lozenges, each containing a predetermined amount of the vector delivery structure; or as a suspension.

The term “comprising” and grammatical variants thereof means “including” or “consisting”. Thus, for example, a composition “comprising” X may consist exclusively of X or may include one or more additional components.

A twelfth aspect of the invention provides a method for vaccinating a subject comprising administering to the subject an amount of a bacterial live vector vaccine sufficient to elicit an immune response wherein the bacterial live vector vaccine is a bacterium according to the eighth aspect of the invention. This may be achieved by administration of the pharmaceutical composition of the eleventh aspect of the invention to the patient.

A thirteenth aspect of the invention provides a bacterium according to the eighth aspect of the invention for use in medicine.

A fourteenth aspect of the invention provides the use of a bacterium according to the eighth aspect of the invention in the manufacture of a medicament for vaccinating a patient.

The polypeptides of the first and second aspects of the invention can be used to screen libraries of compounds in any of a variety of drug screening techniques. Such compounds may modulate (agonize or antagonize) the expression or activity of a polypeptide of the first or second aspect of the invention. Such compounds may have utility in treating bacterial diseases, in particular those mediated by P. aeruginosa or E. coli and/or in controlling biofilms, in particular P. aeruginosa biofilms. The compounds may also find utility in treating unwanted growth of bacteria belonging to various genera including, for instance, Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Enterococcus, Escherichia, Francisella, Haemophilus, Hafnia, Helicobacter, Klebsiella, Legionella, Listeria, Morganella, Moraxella, Proteus, Providencia, Pseudomonas, Salmonella, Serratia, Shigella, Staphylococcus, Streptococcus, Treponema, Xanthomonas, Vibrio, and Yersinia

Accordingly, a fifteenth aspect of the invention provides a method for identifying an agonist or antagonist compound of a polypeptide of the first or second aspect of the invention.

In one embodiment, the method comprises contacting a test compound with a polypeptide of the first or second aspect of the invention and determining if the test compound binds to the polypeptide of the first or second aspect of the invention. The method may further comprise determining if the test compound enhances or decreases the activity of a polypeptide of the first or second aspect of the invention. Methods for determining if the test compound enhances or decreases the activity of a polypeptide of the first or second aspect of the invention will be known to persons skilled in the art and include, for example, docking experiments/software or X ray crystallography.

In one embodiment, the method comprises screening test compounds for their ability to agonize or antagonize the binding of the protein of the first aspect (Phd-like antitoxin protein) of the invention to the protein of the second aspect of the invention (the ParE-like toxin protein).

The polypeptide of the invention that is employed in the screening methods of the invention may be free in solution, affixed to a solid support, borne on a cell surface or located intracellularly.

Test compounds (i.e. potential agonist or antagonist compounds) may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional minietics of the aforementioned.

Test compounds may be isolated from, for example, cells, cell-free preparations, chemical libraries or natural product mixtures. These agonists or antagonists may be natural or modified substrates, ligands, enzymes, receptors or structural or functional mimetics. For a suitable review of such screening techniques, see Coligan et al., Current Protocols in Immunology 1(2):Chapter 5 (1991).

Compounds that are most likely to be good antagonists are molecules that bind to the polypeptide of the invention without inducing the biological effects of the polypeptide upon binding to it.

Potential antagonists include small organic molecules, peptides, polypeptides and antibodies that bind to the polypeptide of the invention and thereby inhibit or extinguish its activity. In this fashion, binding of the polypeptide to normal cellular binding molecules may be inhibited, such that the natural biological activity of the polypeptide is prevented.

In certain of the embodiments described above, simple binding assays may be used, in which the adherence of a test compound to a surface bearing the polypeptide is detected by means of a label directly or indirectly associated with the test compound or in an assay involving competition with a labelled competitor.

Another technique for drug screening which may be used provides for high throughput screening of compounds having suitable binding affinity to the polypeptide of interest (see International patent application W084/03564). In this method, large numbers of different small test compounds are synthesised on a solid substrate, which may then be reacted with the polypeptide of the invention and washed. One way of immobilising the polypeptide is to use non-neutralizing antibodies. Bound polypeptide may then be detected using methods that are well known in the art. Purified polypeptide can also be coated directly onto plates for use in the aforementioned drug screening techniques.

In a sixteenth aspect, the invention provides a method for identifying a compound that is effective to alter the expression of a target polynucleotide which encodes a polypeptide of the first or second aspect of the invention, the method comprising a) exposing a sample comprising the target polynucleotide to a test compound, b) detecting altered expression, if any, of the target polynucleotide.

The method may further comprise: c) comparing the expression of the target polynucleotide in the presence of varying amounts of the compound and in the absence of the compound.

The compound may either increase (agonize) or decrease (antagonize) the level of expression of the gene.

Compounds which may be effective in altering expression of a specific polynucleotide may include, but are not limited to, oligonucleotides, antisense oligonucleotides, ribozymes, triple helix-forming oligonucleotides, transcription factors and other polypeptide transcriptional regulators, and non-macromolecular chemical entities which are capable of interacting with specific polynucleotide sequences. Effective compounds may, for example, alter polynucleotide expression by acting as either inhibitors or promoters of polynucleotide expression.

In various embodiments, one or more test compounds may be screened for effectiveness in altering expression of a specific polynucleotide. A test compound may be obtained by any method commonly known in the art, including chemical modification of a compound known to be effective in altering polynucleotide expression; selection from an existing, commercially-available or proprietary library of naturally-occurring or non-natural chemical compounds; rational design of a compound based on chemical and/or structural properties of the target polynucleotide; and selection from a library of chemical compounds created combinatorially or randomly. A sample comprising a polynucleotide encoding a polypeptide according to the first or second aspect of the invention is exposed to at least one test compound thus obtained. The sample may comprise, for example, an intact or permeabilized cell, or an in vitro cell-free or reconstituted biochemical system. Alterations in the expression of the polynucleotide are assayed by any method commonly known in the art. Typically, the expression of a specific nucleotide is detected by hybridization with a probe having a nucleotide sequence complementary to the sequence of the polynucleotide. The amount of hybridization may be quantified, thus forming the basis for a comparison of the expression of the polynucleotide both with and without exposure to one or more test compounds. Detection of a change in the expression of a polynucleotide exposed to a test compound indicates that the test compound is effective in altering the expression of the polynucleotide.

Compounds effective in altering expression of the polynucleotide may also be identified using an ELISA which measures secreted or cell-associated levels of polypeptide using monoclonal or polyclonal antibodies by standard methods known in the art, and this can be used to search for compounds that may inhibit or enhance the production of the polypeptide from suitably manipulated cells or tissues.

Preferably, the method of the sixteenth aspect of the invention is used to identify a compound which is differential in its effect on expression of a ParE-like protein of the second aspect of the invention and its effect on expression of a Phd-like protein of the second aspect of the invention. Particularly, preferred are compounds which increase the expression of a ParE-like protein of the second aspect of the invention vis-à-vis expression of a Phd-like protein of the second aspect of the invention and compounds which decrease the expression of a Phd-like protein of the second aspect of the invention vis-à-vis expression of a ParE-like protein of the second aspect of the invention. Such compounds are envisaged as being particularly useful in controlling growth of bacteria, such as P. aeruginosa.

In addition to antagonists and agonists of the fifteenth and sixteenth aspect of the invention having utility in modulating bacterial growth, the proteins of the first and second aspects of the invention may also be useful in modulating bacterial growth and biofilms. The proteins may find utility in controlling bacterial growth or biofilms in a wide variety of conditions and circumstances.

Accordingly, a seventeenth aspect of the invention provides a method of modulating cell growth, the method comprising contacting the cells whose growth is to be controlled with a protein of the first or second aspect of the invention.

The method of the seventeenth aspect of the invention may be an in vitro or an in vivo method.

In one embodiment of the seventeenth aspect of the invention there is provided a method of treating or preventing an infection (e.g. a bacterial infection) in a patient in need thereof comprising administering to the patient a protein of the second aspect of the invention.

An eighteenth aspect of the invention provides a protein of the first or second aspect of the invention for use in medicine.

A nineteenth aspect there is provided the use of a protein of the first or second aspect of the invention in the manufacture of a medicament for preventing or treating an infection (e.g. a bacterial infection).

A twentieth aspect of the invention provides a pharmaceutical composition comprising a protein of the second aspect of the invention.

By “modulating growth” we include where there is an increase or decrease in the amount of cells or where there is an increase or decrease in the rate of cell growth as compared with untreated cells. The protein of the second aspect of the invention (i.e. the parE-like protein and functional equivalents thereof etc.) may be used to control (prevent or treat) infections such as bacterial infections. The protein of the second aspect of the invention may also be used in vitro for instance in various industrial settings.

The cells whose growth may be controlled with a protein of the first or second aspect of the invention include bacteria and eukaryotic cells. Examples of eukaryotic cells include: fungi (e.g. Saccharomyces spp., Candida spp.), animal cells (vertebrate or invertebrate), plant cells and protoctistan cells (e.g. protozoa and algal cells). Whilst the toxin protein of the invention exhibits toxicity in respect of bacteria there is some evidence that it may also inhibit growth of eukaryotic cells as well.

A recent publication has indicated that another antitoxin-toxin pair (kis/kid) has effects on eukaryotic cells, retarding growth but not necessarily killing it. Reference is made to: 1) Ramon Diaz-Orejas et al, 2003,ELSO gazette, 17, P1-9 AND 2) de la Cueva-Mendez G et al, 2003, EMBO J, 22; 246-251. Accordingly, it is believed that the polypeptides of the invention may effect eukaryotic cells as well as prokaryotic cells.

In one embodiment of the invention, a protein of the first or second aspect of the invention may be used to modulate the growth of biofilms. A protein of the second aspect of the invention may be used to prevent or delay the initiation of a biofilm infection or to prevent or delay the progression or advancement of a biofilm infection. A protein of the second aspect of the invention may also be used to treat a biofilm, e.g. to reduce its size etc.

The bacteria may be gram negative or gram positive.

In one embodiment the bacteria may be selected from the group consisting of: Aerobacter, Aeromonas, Acinetobacter, Agrobacterium, Bacillus, Bacteroides, Bartonella, Bortella, Brucella, Calymmatobacterium, Campylobacter, Citrobacter, Clostridium, Cornyebacterium, Enterobacter, Enterococcus, Escherichia (e.g. E. coli), Francisella, Haemophilus, Hafnia, Helicobacter, Klebsiella, Legionella, Listeria, Morganella, Moraxella, Proteus, Providencia, Pseudomonas (e.g. Pseudomonas aeruginosa), Salmonella, Serratia, Shigella, Staphylococcus, Streptococcus (e.g. S. pyrogenes), Treponema, Xanthomonas, Vibrio, and Yersinia

A protein of the second aspect of the invention may be used to treat or prevent an infection, for instance an infection selected from the group consisting of: urinary tract infections, respiratory system infections, dermatitis, conjunctivitis, otitis, skin and soft tissue infections, bacteraemia, bone and joint infections, gastrointestinal infections, eye infections, ear infections, and endocarditis.

A protein of the second aspect of the invention may find particular utility in treating or preventing Pseudomonas aeruginosa infections. This bacterium is implicated in many infections. For instance, Pseudomonas aeruginosa colonization in the eye leads to bacterial keratitis or corneal ulcer and endophthalmitis. Also, Pseudomonas aeruginosa is a common bacterium residing in the ear canal and is a common pathogen causing external otitis. Pseudomonas aeruginosa is a common causative agent in complicated and nosocomial urinary tract infections. Opportunities for infection occur during catheterization, surgery, obstruction and blood-borne transfer of Pseudomonas aeruginosa to the urinary tract. Pseudomonas aeruginosa can also cause opportunistic infections in skin and soft tissue in locations where the integrity of the tissue is broken by trauma, burn injury, dermatitis and ulcers resulting from peripheral vascular disease. Pseudomonas aeruginosa has been shown to have a high affinity to cardiac tissue including heart valve tissue.

In one embodiment, a protein of the second aspect of the invention is used to prevent or treat lung infections, for instance lung infections in cystic fibrosis patients.

In one embodiment, a protein of the second aspect of the invention may be used to control bacterial growth on medical devices.

Biofilms containing pathogenic bacteria such as Pseudomonas aeruginosa can form on a variety of devices used in biomedical research and clinical care, including endrotracheal tubes used for chronic mechanical ventilation, indwelling catheters, vascular prostheses, cardiac pacemakers, prosthetic heart valves, biliary stents, indwelling urinary catheters, chronic peritoneal dialysis catheters, extended-wear contact lenses, and artificial joints, resulting in serious infections which are unresponsive to antimicrobial therapy.

Biofilm infections of indwelling devices such as prosthetic joints, heart valves, and catheters are among the most serious and difficult infections to eradicate. Often, the device must be removed to cure the infection.

In one embodiment of the invention, the biofilm to be treated is formed on an indwelling device. As used herein, “indwelling device” includes any device left within the body for an extended period of time such as a catheter or prosthesis. In a specific embodiment, the biofilm is formed on a prosthetic device. In another embodiment the biofilm is formed on a catheter.

It is envisaged that the proteins of the second aspect of the invention may find utility in the care of a wide variety of patients. The patient may, for example, be a mammal such as a human.

In one embodiment the patient is a burns patient, a cancer patient, a cystic fibrosis patient or an HIV/AIDS patient. Such patients may be particularly susceptible to infections.

The pharmaceutical compositions of the invention may include pharmaceutically effective carriers, and optionally, may include other therapeutic ingredients, such as various adjuvants known in the art or antibiotics etc.

The carrier or carriers must be pharmaceutically acceptable in the sense that they are compatible with the therapeutic ingredients and are not unduly deleterious to the recipient thereof.

Suitable carriers, adjuvants, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

An adjuvant is a substance that increases the immunological response of the subject (e.g. human) to the vaccine. Suitable adjuvants include, but are not limited to, aluminum hydroxide (alum), immunostimulating complexes (ISCOMS), non-ionic block polymers or copolymers, cytokines (like IL-1, IL-2, IL-7, IFN-α, IFN-β, IFN-γ, etc.), saponins, monophosphoryl lipid A (MLA), muramyl dipeptides (MDP) and the like. Other suitable adjuvants include, for example, aluminum potassium sulfate, heat-labile or heat-stable enterotoxin isolated from Escherichia coli, cholera toxin or the B subunit thereof, diphtheria toxin, tetanus toxin, pertussis toxin, Freund's incomplete or complete adjuvant, etc. Toxin-based adjuvants, such as diphtheria toxin, tetanus toxin and pertussis toxin may be inactivated prior to use, for example, by treatment with formaldehyde.

Furthermore, the antigen or immunogen may be conjugated to a bacterial toxoid, such as a toxoid from diphtheria, tetanus, cholera, H. pylori, and other pathogens.

By employing an adjuvant or otherwise providing a protein of the second aspect aspect of the invention in a form or formulation with allows development of an immune response in a patient it may be possible for an immune response to develop in the subject/patient which enables the development of an immune response against a polypeptide of the second aspect of the invention. This may be useful in enabling the patient to mount an immune response against the pf4 bacteriophage. In an alternative embodiment of the invention a polypeptide of the first aspect of the invention could be employed as an immunogen although it will of course be appreciated that administration of a polypeptide of the invention would be non-toxic to the target cells to be controlled whereas a polypeptide of the second aspect of the invention would have the advantage of being toxic.

The effective amount of a protein of the invention to be administered to a patient will depend upon a number of factors, for instance the severity of the infection, general health of the subject, age, weight, and gender of the subject, diet, time and frequency of administration, drug combination(s), reaction sensitivities, and tolerance/response to therapy. The effective dose for a given situation can be determined by routine experimentation and is within the judgement of the skilled person. Compositions may be administered individually to a patient or may be administered in combination with other agents or drugs.

For example, in order to formulate a range of dosage values, cell culture assays and animal studies can be used. The animal model may also be used to determine the appropriate concentration range and route of administration. Such information can then be used to determine useful doses and routes for administration in humans. The dosage of such compounds preferably lies within the dose that is therapeutically effective in 50% of the population, and that exhibits little or no toxicity at this level. The dosage should of course be such that the bacterial growth is modulated by a useful amount.

Dosage treatment may be a single dose schedule or a multiple dose schedule.

Administration of the compositions of the invention may be effected by different ways, e.g., by oral, intravenous, intraperitoneal, subcutaneous, intramuscular, topical or intradermal administration. Topical administration may, for example, be achieved by providing the pharmaceutical composition in the form of a wash solution, cream, or in the form of a wound dressing.

The pharmaceutical compositions can in one embodiment be infused or otherwise delivered into any fluid, tissue or structure of the body including but not limited to the blood, tissues, cerebral spinal fluid (CFS), eye, oral cavity, peritoneum, pleural spaces, and/or joints of patients infected with bacteria or which are susceptible to bacterial infection.

The compositions of the invention may be administered locally or systemically.

Compositions of the present invention can be presented, for example, as discrete units such as capsules, cachets, tablets or lozenges, each containing a predetermined amount of the active ingredient; or as a suspension.

The proteins of the present invention may also be employed to modulate cell (e.g. bacterial) growth in a number of in vitro settings. For instance, unwanted Pseudomonas aeruginosa growth is associated with a wide variety of industrial, commercial and processing operations such as sewerage discharges, recirculating water systems (cooling tower, air conditioning systems etc.), water condensate collections, paper pulping operations and, in general, any water bearing, handling, processing, collection etc. systems.

The proteins of the present invention can, for example, be made into solution with a combination of one or more sanitizers and/or one or more enzymes that will facilitate penetration and break down of the matrix improving efficiency.

In one embodiment proteins of the second aspect of the invention may be used in cleaning, disinfecting, or decontaminating a surface, the method comprising contacting the surface with a cleaning composition comprising a protein of the second aspect of the invention.

Where the proteins of the invention are used to modulate bacterial growth (whether in vivo or in vitro) and the bacteria are present in the form of a biofilm it may be advantageous to at least partially dismantle the biofilm. The biofilm may be at least partially dismantled prior to, during and/or after application of the protein of the invention. (e.g. using enzymes or bacteriophages such as Pf4 or even sonication). As mentioned above, biofilm cells have been shown to be 500 times more resistant to antibacterial agents as compared to planktonic forms. Accordingly, dismantling the biofilm may result in an increase in the efficacy of the proteins and compositions of the invention.

To effect dismantling of biofilms, enzymes (e.g. alginate lyase, carboxylic ester hydrolases, sulfuric ester hydrolases, glycosidases and lyases acting on polysaccharides.) which can degrade biofilms and other techniques/moieties may be employed which will be known or can be readily devised by those skilled in the art. In this regard, the teachings of WO0193875 are incorporated herein by reference.

Agents for dismantling biofilms may be provided in the form of a kit with a protein or composition of the invention. Such kits form a further aspect of the invention.

The practice of the present invention will employ, unless otherwise indicated, conventional techniques of molecular biology, microbiology, and recombinant DNA technology which are within the skill of those working in the art. Such techniques are explained fully in the literature. Examples of texts for consultation include the following: Sambrook Molecular Cloning; A Laboratory Manual, Third Edition (2000).

### EXAMPLES

### Example 1

A current question in biofilm research is whether biofilm-specific genetic processes can lead to differentiation in physiology and function among biofilm cells. In Pseudomonas aeruginosa, phenotypic variants which exhibit a small colony phenotype on agar media, and which demonstrate a markedly accelerated pattern of biofilm development when compared to the parental strain, are often isolated from biofilms. We grew P. aeruginosa biofilms in glass flow-cell reactors and observed that the emergence of small colony variants (SCV's) in the effluent run-off from the biofilm correlated with the emergence of plaque-forming Pf1-like filamentous phage (here designated Pf4) from the biofilm. Because several recent studies have highlighted that bacteriophage genes are among the most highly upregulated groups of genes during biofilm development, we investigated whether Pf4 plays a role in SCV formation during P. aeruginosa biofilm development. We carried out immunoelectron microscopy using anti-Pf4 antibodies and observed that SCV cells, but not parental-type cells, exhibited high densities of Pf4 filaments on the cell surface, and that these filaments were often tightly interwoven into complex ‘latticeworks’ surrounding the cells. Moreover, infection of P. aeruginosa planktonic cultures with Pf4 caused the emergence of SCVs within the culture. These SCVs demonstrated enhanced attachment, accelerated biofilm development, and large regions of dead and lysed cells inside microcolonies, in an identical manner to SCVs obtained from biofilms. We conclude that Pf4 can mediate phenotypic variation in P. aeruginosa biofilms. We also carried out partial sequencing and analysis of the Pf4 replicative form and have identified a number of open reading frames not previously recognised within the genome of P. aeruginosa, including a post-segregational killing operon.

Bacteria in biofilms often form densely-packed, matrix-encased structures (microcolonies) in which steep oxygen and nutrient availability gradients can occur (10, 53). Bacterial adaptation to such highly heterogeneous and changing conditions is thought to include the development of phenotypic variants, which may become established as niche specialists within the biofilm (2, 43, 48). One such example is the significant colony variation observed among P. aeruginosa cells obtained from laboratory biofilms, as well as from persistent clinical infections caused by P. aeruginosa. Such variants include mucoid (11, 37), dwarf (18, 19, 37), lipopolysaccharide deficient (9), rough (37), hyperpiliated (12, 19) and antibiotic resistant (14) colonies. Although much remains to be learned about the processes that cause phenotypic variation within biofilms, they may reflect inducible mechanisms that generate genetic variability under conditions of stress or significant environmental change.

Bacteria possess diverse mechanisms that may lead to an increase in genetic and phenotypic variability under conditions of stress. These processes include adaptive mutation (3, 43, 55), phase variation (14) and enhanced gene transfer through processes of conjugation and transformation (17, 20, 41). In addition, the relationship between bacterial stress responses and the mobility of bacteriophages has been extensively documented, and bacteriophage transduction is now increasingly recognized for its importance in gene transfer within natural bacterial populations (40). Moreover, bacterial prophages can cause DNA inversions and phenotypic variation (32, 57) and bacteria often acquire phenotypic traits, such as virulence factors (13), from the genome of an infecting bacteriophage.

Recent studies have highlighted bacteriophage genes as being among the most highly up-regulated groups of genes during biofilm development in both Gram positive and Gram negative bacteria (52, 63). In P. aeruginosa, genes of a Pf1-like filamentous bacteriophage, which exists as a prophage within the genome of P. aeruginosa, showed up to 83.5-fold activation during biofilm development, compared with planktonic cells (63). Other studies have shown that activation of Pf1 genes in biofilms is regulated by quorum-sensing in P. aeruginosa (21, 58). Moreover, activity of the Pf1-like phage is also linked to the killing and lysis of a subpopulation of P. aeruginosa cells within biofilms (59). Induction of the Pf1-like phage (here designated Pf4) in P. aeruginosa may therefore represent an important physiological and developmental event during biofilm development.

Here we show that activity of the Pf4 phage in P. aeruginosa biofilms is linked to the emergence of a subpopulation of cells with a small-colony phenotype in the effluent run-off from the biofilm. These cells exhibit high densities of filamentous phage on the cell-surface, demonstrate enhanced adhesion and microcolony development, and occur in high numbers within the biofilm run-off. Our data suggest that Pf4-SCVs play an important role in biofilm development, as well as in the colonization of new surfaces during biofilm dispersal.

### Materials and Methods

Strains and culture conditions. Pseudomonas aeruginosa strain PAO1 (26) was used in this study. Batch cultures of P. aeruginosa were grown at 37° C. with shaking in Luria Bertani (LB) medium. For cultivation of biofilms, M9 medium containing 48 mM Na₂HPO₄, 22 mM KH₂PO₄, 9 mM NaCl, 19 mM NH₄Cl, 2 mM MgSO₄, 100 μM CaCl₂, and 5 mM glucose was used.

Biofilm experiments. P. aeruginosa PAO1 wild-type and small colony variant biofilms were grown in continuous-culture flow-cells (channel dimensions 1×4×40 mm) at room temperature as previously described (42). Channels were inoculated with 0.5 ml of early stationary phase cultures containing approximately 1×10⁹ cells ml⁻¹ and incubated without flow for 1 h at room temperature. Flow was then started with a mean velocity in the flow cells of 0.2 mm s⁻¹, corresponding to laminar flow with a Reynolds number of 0.02. Biofilms were stained with using the LIVE/DEAD BacLight Bacterial Viability Kit (Molecular Probes Inc., Eugene, Oreg.) and visualized using a confocal laser scanning microscope (CLSM) (Olympus). The two stock solutions of the stain (SYTO 9 and propidium iodide) were diluted to 3 μl ml⁻¹ in biofilm medium and injected into the flow channels. Live SYTO 9-stained cells and dead propidium iodide-stained cells were visualized with a confocal laser scanning microscope (CLSM) (Olympus) using fluorescein isothiocyanate and tetramethyl rhodamine isocyanate optical filters, respectively. For isolation of colony variants, 1 ml aliquots of effluent biofilm run-off (spent culture medium emerging from the flow cell reactor, and containing detached biofilm cells) were collected after 1, 3, 5, 7 and 9 days of biofilm development. This effluent was then serially diluted from 10⁻² to 10⁻⁶ and plated onto LB agar. Plates were observed for colony variants after 48 h incubation at 37° C.

To provide statistically based, quantitative measurements during biofilm development, we characterized biofilm morphology by using the COMSTAT program (23). Biofilms were stained with acridine orange (ProSciTech, Kelso, Australia). At each time point, 5 image stacks were recorded for 3 replicate biofilms, resulting in 15 image stacks for each strain studied. Images were acquired at 2 μm intervals through the biofilm at random positions in the flow cell at 3 time points (1, 3, and 7 days) as previously described (23) by using CLSM. The following parameters were assessed: total biovolume (μm³ μm⁻²), maximum thickness (μm), average thickness (μm), and average microcolony area at the substratum (μm²).

Adhesion assay. We compared the ability of WT and SCV strains to adhere to wells of polystyrene micro-titer plates using an adhesion assay similar to that previously described (45). P. aeruginosa and SCV cultures were grown to OD₆₀₀ 0.6. Cells were centrifuged (6000×g for 15 min) and resuspended to an OD₆₀₀ reading of 0.1. Aliquots (200 μl) of cells were then placed into 96 well micro-titre plates and incubated for 2 hours at 37° C. After this time 25 μl of a 1% solution of crystal violet (CV) was added to each well, the plates were incubated at room temperature for 15 min and rinsed three times with water. Ethanol (200 μL) was then added to each well to extract the CV, and the extent of CV staining was measured using an ELISA plate reader (Wallac, Perkin Elmer) at 600 nm.

Bacteriophage experiments. Isolation of Pf4 plaque forming units (PFU) from biofilms, determination of phage titers, and large scale preparation and purification of Pf4 were carried out as described previously (59). Infection of planktonic P. aeruginosa cultures with Pf4 was carried out at a multiplicity of infection (MOI) of 10. For the preparation of replicative form (RF) DNA of Pf4, a 1 L early log-phase culture of P. aeruginosa PAO1 was infected with Pf4. After 12 h incubation with shaking (150 revs min⁻¹), cells were harvested by centrifugation at 7,000 rpm and the supernatant discarded. Cell pellets were washed with sodium chloride Tris-EDTA (STE; 100 mM NaCl, 10 mM Tris, 1 mM EDTA, pH8) and then resuspended in 20 ml of 10 mM Tris (pH 8.0) solution. Cell lysis and extraction of RF DNA was performed with using a Qiagen Maxiprep kit (Qiagen, Germany). The final pellet was resuspended in 150 μl of TE (Tris-EDTA; 10 mM Tris, 1 mM EDTA, pH8), visualised on an agarose gel, and the 12 kb RF band was excised from the gel and purified using a QIAquick kit (Qiagen).

To confirm the production of the Pf4 RF, and to accurately delineate Pf4 within the P. aeruginosa PAO1 genome, we amplified a region of the RF predicted to contain the region of recircularization of the replicative form (RF), i.e. containing the direct repeats which flank the predicted phage genome (28). Thus we designed primers that would amplify a product only from the Pf4 RF, and not from the Pf4 prophage within the genome of P. aeruginosa. Primers Pf4F (5′-AGCAGCGCGAT GAAGCAAT-3′), corresponding to bp 2756 to 2774 of GenBank accession no. AE004508, and Pf4R (5′-TAGAGGCCAT TTGTGACTGGA-3′), targeting bp 1566 to 1546 of GenBank accession no. AE004507 were used for this purpose. The 839 bp PCR product was purified by using a Qiagen PCR cleanup kit (Qiagen, USA) and sequenced using the BigDye® termination reaction (Applied Biosystems, Australia) and an ABI 3730 sequencer. For analysis of the Pf4 genome (obtained from the P. aeruginosa PAO1 genome sequence) and the Pf1 genome, sequences were compared using the National Center for Biotechnology Information (NCBI) BLAST and ORF Finder programs.

Preparation of antibodies and immunolabeling. Anti-Pf4 polyclonal antibodies were developed using a synthetic peptide (Auspep, Australia) with the following amino acid sequence: Gly-Val-Ile-Asp-Thr-Ser-Ala-Val-Glu-Ser-Ala-Ile-Thr-Asp-Gly-Cys. This sequence corresponds to residues 1 to 15 of CoaB (PA0723; the major coat protein of the filamentous phage virion) of Pf1 (and of Pf4), which is exposed on the outer surface of the bacteriophage virion (35, 60). An extra cysteine residue was added to the N-terminus of this peptide for coupling to the carrier protein, keyhole limpet haemocyanin (KLH). A rabbit was immunized by using 3 sub-cutaneous injections, each containing 300 μg of the KLH-conjugated CoaB peptide (Institute of Medical and Veterinary Science, Adelaide, Australia). The serum titre and specificity of the polyclonal antibodies was monitored by ELISA and Western blot analysis.

Electron microscopy and immunogold labeling of Pf4 virions. Immunogold electron microscopy was carried out on SCV and wild-type cells grown on agar plates for 18 h at 37° C., essentially as described (50). Bacteria scraped from the agar surface with a cotton swab were suspended in phosphate-buffered saline (PBS). A drop (50 μl) of this suspension was placed onto a sheet of Parafilm. A carbon and Formvar-coated nickel grid was placed on the drop, with the coating facing the drop, for 2 min and then sequentially onto drops on the following reagents (at room temperature): PBS containing 0.1% glutaraldehyde (5 min), PBS containing 50 mM NH₄Cl (5 min), PBS containing 1% bovine serum albumin (BSA) and 1% normal goat serum (NGS) (5 min), and then rabbit anti-Pf4 antiserum diluted 1/100 in PBS1% BSA, 1% NGS (30 min). After three washes in PBS 0.1% BSA (2 min each), the grid was placed on a drop of immunoglobulin-gold-conjugated anti-rabbit immunoglobulin G (IgG) (heavy and light chains) (12 nm-diameter gold particles; Jackson Immunoresearch) diluted in PBS (30 min). The grids were subjected to three washes in PBS (3 min each), fixed in 1% glutaraldehyde in PBS (5 min), and washed twice in distilled water (for 5 min each). The grids were then treated with a drop of 1% uranyl acetate for 30 s. Grids were air dried and examined using a Hitachi H7000 Transmission Electron Microscope (TEM).

### Results and Discussion

Emergence of SCVs within the Biofilm Effluent Correlates with the Release of Plaque-Forming Pf4 Phage Variants.

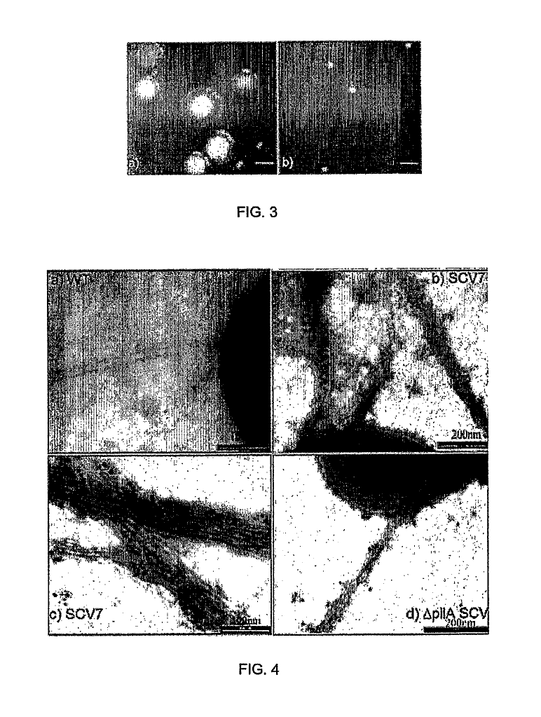

Previously, genes of a Pf1-like filamentous prophage were found to be highly upregulated during P. aeruginosa biofilm development (63), and mature biofilms of Pseudomonas aeruginosa were found to release a filamentous phage (here designated Pf4) capable of forming plaques on the host strain of P. aeruginosa (59). Because filamentous phage infection can cause small colonies in E. coli cultures (30), we hypothesized that filamentous phage may also be important in the formation of P. aeruginosa SCVs during biofilm development. We therefore compared colony-forming unit (CFU) and phage plaque-forming unit (PFU) counts on agar plates using the effluent run-off from P. aeruginosa biofilms (Table 1). For the first 5 days of biofilm development we observed between 1.3×10⁶ and 3.7×10⁶ CFU ml⁻¹ effluent, and all of these colonies resembled those normally formed by the parental strain P. aeruginosa. No phage were detected in the effluent during this period. However, after 7 days, we observed the simultaneous emergence of 1×10⁵ SCVs ml⁻¹, and 1×10⁷ PFU ml⁻¹ of phage Pf4, alongside 3.0×10⁶ CFU ml⁻¹ of the wild-type large colonies. Colonies of the SCVs were approximately 0.5-1.5 mm in diameter, whereas parental-type colonies were >4 mm in diameter (FIG. 3a).

We picked colonies from the 18 h agar plates and found that all of the SCVs, but not wild-type colonies, contained superinfective (able to form plaques on the lysogenic P. aeruginosa strain) Pf4 bacteriophage (approx. 1×10⁷ PFU ml⁻¹). To confirm that these SCVs were variants of P. aeruginosa PAO1, and not a contaminant, we sequenced regions of the 16S rDNA of these variants, and found 100% identity with the P. aeruginosa genome sequence (54). We selected one variant, designated SCV7, for subsequent investigation.

Immunoelectron microscopy reveals dense ‘latticeworks’ of Pf4 filaments surrounding SCV cells. Because SCV colonies contained high numbers of Pf4, we expected that electron microscopic examination would reveal high densities of Pf4 filaments on the surface of SCV cells compared to the wild type strain. We therefore carried out immunoelectron microscopy of P. aeruginosa cells from normal and SCV7 colonies, using antibodies raised against the Pf4 major coat protein. SCV7 colonies, but not wild-type colonies, contained cells that were surrounded by high densities of Pf4 filaments (FIG. 4). The original immuno-electron microscopic descriptions of filamentous phage Pf in P. aeruginosa by Bradley (4) also demonstrated phage filaments that were often tightly interwoven in ‘skeins’, identical to our observations.

Previously, small colony variants that emerge from P. aeruginosa biofilms have been reported to overproduce type IV pili on the cell surface (12, 19). Type IV pili are structurally very similar to filamentous phage virions and they are often indistinguishable using electron microscopy. To provide further evidence that the hyperfilamentation observed in this study was principally due to Pf4 filaments, we grew biofilms using a P. aeruginosa mutant with a knock-out insertion in pilA which codes for the major structural subunit of the type-IV pilus (29). We found that mature ΔpilA biofilms also produced SCVs that contained Pf4 PFUs, and exhibited similar high densities of surface Pf4 filaments when examined by immunoelectron microscopy (FIG. 4d). These data suggest that Pf4 virions are the principal cause of hyperfilamentation in SCV7.

Addition of Pf4 virions to P. aeruginosa cultures generates SCVs. To further investigate the role of Pf4 in SCV formation, we infected planktonic cultures of P. aeruginosa with CsCl gradient-purified Pf4. After 12 h incubation in the presence of the phage, we found that all of the cells within the culture grew with a small-colony phenotype (FIG. 3b), whereas uninfected cultures produced normal sized colonies. These SCVs (designated Pf4-SCVs) were identical in appearance to SCVs that emerged from the biofilm, and also exhibited high densities of filamentous phage on the cell surface in an identical manner to that of SCV7 (data not shown).

How might filamentous phage arise and cause an altered colony phenotype in the host bacterium? Wild-type Pf4, like other filamentous phage, establishes a symbiotic state with its host and is continuously released from P. aeruginosa cells under normal culture conditions (59, 63). These wild-type phage do not form visible plaques, have little effect on growth of the lysogenised P. aeruginosa host strain, and do not generate SCVs. Thus SCVs are not formed by induction of the wild-type phage. However, filamentous phage that can overcome the lysogenic immunity of the host strain often spontaneously arise in infected cultures (9, 17, 32, 35, 46). Such mutants can cause marked decreases in cellular DNA, RNA and protein synthesis (25, 26, 35), can kill over 60% of the infected cells (31), and result in a colony size that is considerably smaller than that of the uninfected cells (31). Phage that emerge from mature P. aeruginosa biofilms may represent such variant forms of Pf4, and P. aeruginosa cells that can propagate variant phage without being killed likely form SCVs. However, the mechanism of lysogenic immunity toward Pf4, and the mechanism by which spontaneous phage-variants may overcome this immunity, are unknown.

We expected that SCVs would grow more slowly than the wild-type strain in planktonic culture. In fact, SCVs exhibited similar growth rates to the wild-type strain (mean doubling times for the wild-type and SCV7 strains during early logarithmic growth were 47.4 and 43.2 min respectively), thus the SCV phenotype is not caused by slower growth of Pf4-infected cells. Small colonies could also be produced if SCV cells adhere more tightly to one-another compared to the wild-type strain. Indeed, aggregation of cells in SCV planktonic cultures could normally observed by eye (data not shown), and SCV cells were found to be more adherent to wells of microtitre plates (see below).

### Biofilms Formed by SCVs and by Pf4-Infected Cells Show Enhanced Attachment and Microcolony Development.

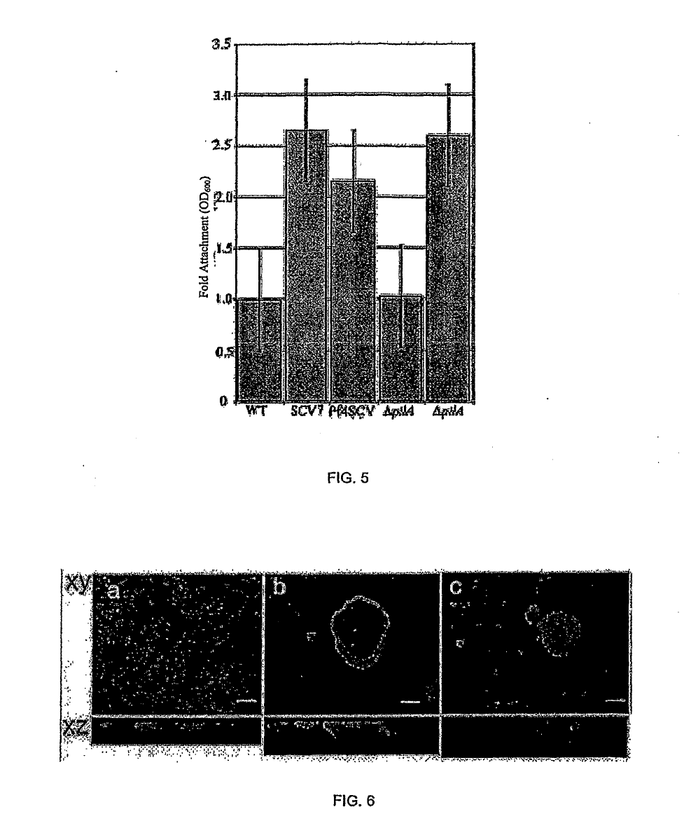

We examined the ability of wild-type, ΔpilA, SCV7, ΔpilA-SCV and Pf4-SCV strains to attach to an inanimate surface. SCV strains each showed increased attachment to the wells of polystyrene micro-titre plates by two-fold or more (FIG. 5). Enhanced attachment of the ΔpiLA-SCV compared to the ΔpilA strain suggests that type-IV pili are not responsible for the increased attachment observed in this study.

The mechanism by which filamentous bacteriophages may lead to increased surface attachment and autoaggregation is unclear. During bacterial attachment, an energy barrier (known as the secondary minimum) is presented to cells whereby electrostatic repulsion can prevent closer approach of the cell to the surface (7). Because of their extremely small radii, cell surface filaments such as type-IV pili can extend through this energy barrier and facilitate bridging and permanent attachment of cells to the surface (36). The copious production of ‘interwoven’ phage filaments could allow for a large number of phage filaments to be in contact with the bacterial cell surface at any one time. Possibly, high numbers of Pf4 with low affinity binding to the substratum or other bacterial cells could result in enhanced adhesion as described for type-IV pili or other cell surface filaments.

We also grew biofilms of the SCVs in continuous culture in glass flow cells. Both the biofilm (SCV7) and planktonically-derived SCVs were capable of forming much larger attached microcolonies than the wild-type strain (FIG. 6). In the wild type strain, the size of microcolonies did not exceed 75 μm in diameter at any stage during biofilm development. In contrast, after five days of biofilm development, SCVs frequently formed microcolonies in the range 200-300 μm in diameter (FIG. 6b,c).

We also compared biofilm development in wild-type and phage-expressing cells using the COMSTAT software (23). The results of this analysis are shown in Table. 2. Maximum biofilm thickness and mean microcolony area were significantly higher in the SCV7 strain than in the wild-type strain at each of the time points studied. SCVs obtained from Pf4-infected planktonic cultures also showed significantly increased maximum biofilm thickness and microcolony area after 3 days of biofilm development. While not always significantly higher using analysis of variance, mean biovolume and thickness were also consistently higher for SCVs compared to the wild-type throughout biofilm development (Table 2).

In biofilms formed with SCVs, we also observed complex heterogeneity within the microcolonies, which contained large regions of dead and lysed cells, as well as hollow voids that did not contain cells. The killing and lysis within microcolonies occurred much earlier in SCV biofilms (4-5 days) than in wild-type biofilms, which occurs after 7 days as observed previously (59). Pf4 has previously been linked with the death and lysis of a subpopulation of cells inside microcolonies in mature (7-day) P. aeruginosa biofilms (59). Filamentous bacteriophages can kill a proportion of host cells (25, 30, 31, 34, 46, 51). The consequences of this cell death to surviving cells within the biofilm, and to the propagation and dispersal of the bacteriophage, remain to be fully elucidated. For example, it is possible that nutrients released by cell death in this manner are assimilated by other bacteria in the biofilm (49, 56).

Several mechanisms may explain how Pf4 activity leads to the formation of larger, more differentiated colonies than the wild-type strain. We observed that SCV7 and phage producing strains were unable to carry out type-IV pilus-mediated twitching motility (data not shown), possibly because filamentous phages use the type-IV pilus as the receptor for infection (38) and might therefore interfere with pilus function. Previously, mutant P. aeruginosa strains incapable of twitching motility were found to form larger microcolony structures than the wild-type strain (22, 29). However, other studies have found that type-IV pilus mutants were unable to form microcolonies (44), thus the role of type-IV pili in microcolony development appears to vary depending on the experimental system used. In this study, biofilms formed by the ΔpilA strain exhibited microcolonies similar to the wild-type strain (data not shown), thus impaired twitching motility is unlikely to be the cause of enhanced microcolony development in SCV strains.

Another mechanism for enhanced microcolony formation is that high densities of filamentous phage on the cell surface may play a direct role in the cohesion of biofilm cells. In E. coli, expression of conjugative plasmid-encoded pili can lead to enhanced microcolony and biofilm formation (16, 47). This process is thought to facilitate plasmid maintenance within the population by allowing high rates of infectious transfer (16). Filamentous bacteriophages share striking functional similarities with conjugative plasmids; indeed several authors have suggested that conjugative pili may have evolutionary links with filamentous bacteriophages (1, 5, 61). Because biofilm formation would similarly enhance the maintenance and infectious transfer of bacteriophages within the host cell population, it is interesting to consider whether the cohesion of biofilm cells by filamentous structures may have its evolutionary origins among the filamentous bacteriophages.

### Analysis of the Pf4 Genome.

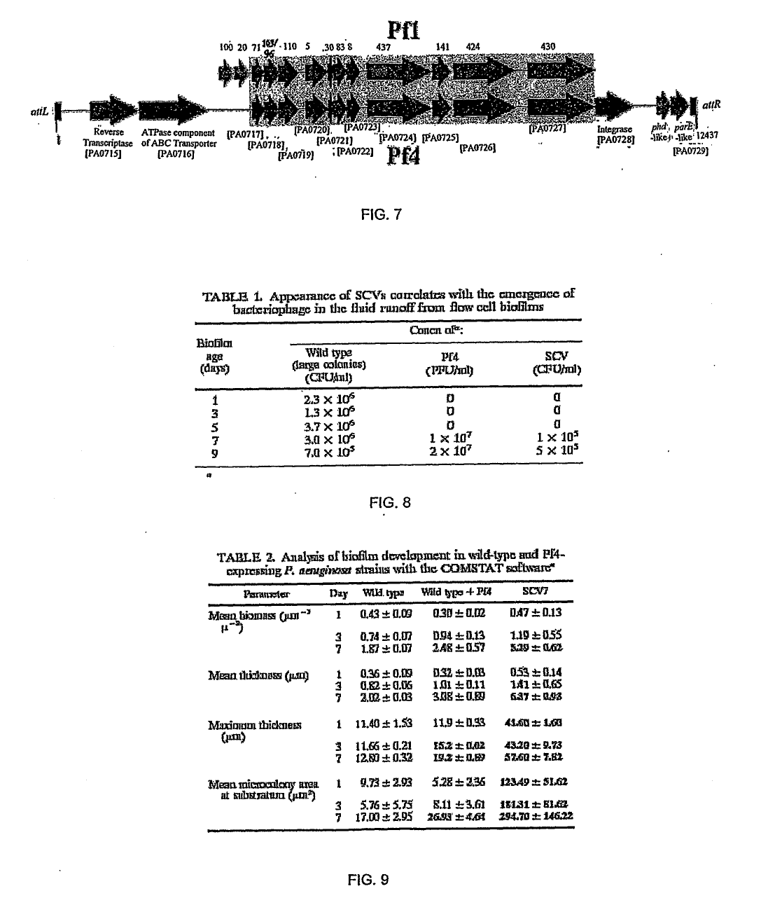

A previous study by our laboratory (59), and the present study confirm the production of virions encoded by the Pf4 prophage of P. aeruginosa. To provide more information about the genome of Pf4, we extracted the RF plasmid from Pf4-infected cells, and used primers Pf4F and Pf4R to amplify the predicted region of recircularization of the phage genome (8, 28) from the RF DNA. Our data confirm that the RF recircularizes using the repeat sequence: TGGAGCGGGCGAAGGGAATCGAACCCT located in the intergenic sequence between PA0714 and PA0715, and within the tRNA-Gly site of PA0729.1. The sequences predict a 12,437 bp viral genome, based on analysis of the published genome sequence of P. aeruginosa (54). A comparison of the genomes of Pf1 and Pf4 is shown in FIG. 7. Major differences include the presence of a putative reverse transcriptase (PA0715), and an ATPase component of an ABC transporter (PA0716) in the Pf4 genome. On the complementary intergenic region between PA0716 and PA0717 (ORF71), is an ORF with 42% homology to the repressor C protein of phage P2 (GenBank accession no. WPBPP2). We also report two ORFs, 9 base pairs apart from one another, encoding proteins with high homology to the prevent-host-death (Phd) antitoxin protein of Pseudomonas syringae (79% identity, Genbank accession no. NP 790091.1) (6), and the conserved domain of the ParE plasmid stabilization toxin of broad host range plasmid RK2 (84.7% identity, Genbank CD no. COG3668.1) (27). These genes are similar in their size and organization to other described toxin-antitoxin modules (15) and suggest that Pf4 may have acquired a host-addiction module, or ‘programmed cell death’ operon. These modules can facilitate the maintenance of plasmids (27) and phages (33) by killing cells that have lost the plasmid or phage post-segregation. To our knowledge, a chromosomally located toxin-antitoxin system has not previously been described in Pseudomonas aeruginosa, and we are currently exploring whether this module is functionally expressed in P. aeruginosa biofilms.

In summary, this study reports a role for the P. aeruginosa prophage Pf4 in the development of small colony phenotypic variants during biofilm formation. These variants exhibit high densities of phage filaments on the cell surface and demonstrate enhanced attachment (2-3 fold) and microcolony development. Bacteriophage-mediated SCV's may represent an important dispersal phenotype, with enhanced colonization traits, which can originate from established P. aeruginosa biofilms in natural environments. Further studies are also needed to determine the significance of high densities of Pf4 filaments as a structural component within the matrix of both laboratory and clinical P. aeruginosa biofilms. Recently, extracellular DNA has been reported as an important component of the extracellular matrix in P. aeruginosa (39, 62). Release of high numbers of DNA-containing Pf4 phage within biofilms may provide one mechanism by which DNA can accumulate in the extracellular matrix of P. aeruginosa biofilms.

### Example 2

### Cloning of the Pare-Like Toxin Gene

White colonies picked up from the pGEM T-Easy-parE transformation, where cultured in LB/Ampicilin at 37° C. overnight. Plasmid DNA was extracted, and the presence of insert DNA was determined by restriction digestion of pGEM T-Easy-parE with restriction enzymes HindIII and PstI and then running gel electrophoresis.

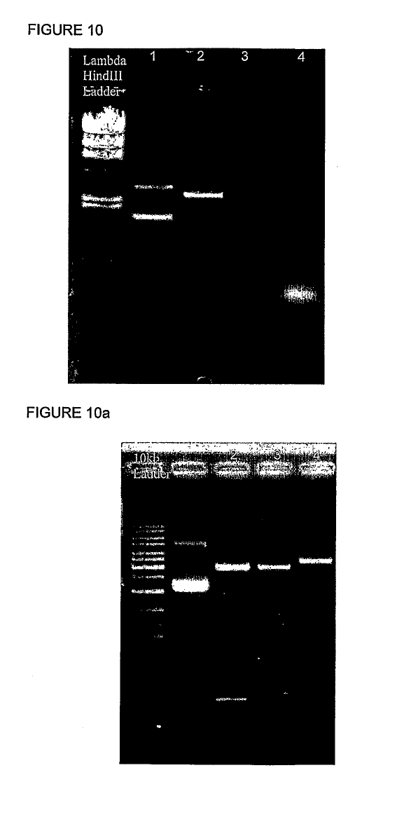

FIG. 10 is the gel picture of the plasmid DNA from the 2 typical positive clones picked from pGEM parE-like clones. Bands observed, from digestion experiments on the plasmid extracted from a typical positive clone confirm that the upper band from pGEM parE-like clone 2 was the digested pGEM vector (3 kb in size). The lower band of pGEM parE-like clone 2 are of size 300 to 400 bp which corresponds to the size of parE-like gene, 348 bp.

Verification of the orientation of insert by restriction mapping was performed. It can be seen from the gel that lane 2 has a fragment of around 3 Kb and a fragment of about 300 to 400 bp. Lane 3 has a band around 3 Kb and a band just below 400 bp. Lane 4 show a fragment size of about 3.5 Kb. This can be observed in FIG. 10a. The data indicates that parE has been successfully cloned and is in the orientation that is down stream of the SP6 promoter.

### Example 3

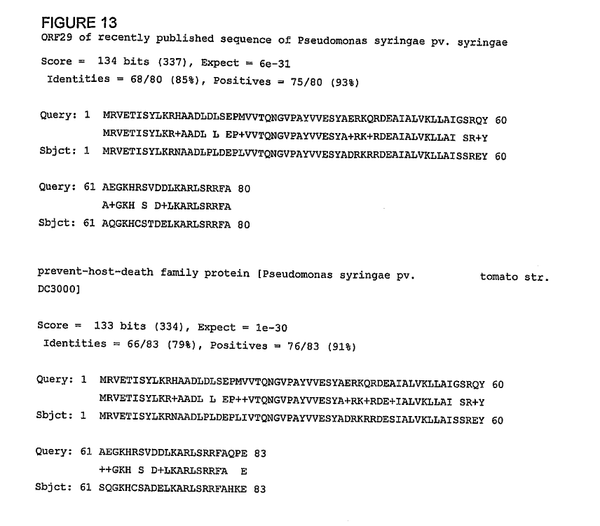

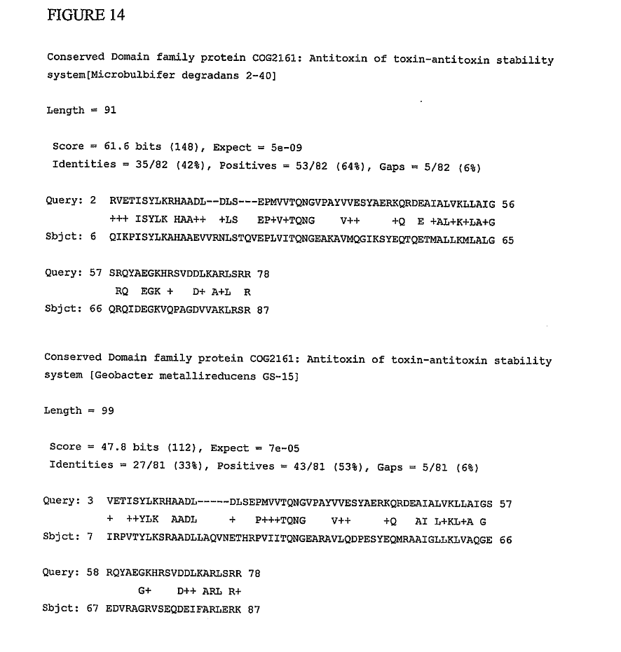

The sequence for the 2 genes (SEQ ID NO.1 and SEQ ID NO.2) and the sequence comparisons with related gene families are as shown in FIGS. 1 & 2. Comparisons with other related proteins are shown in FIGS. 11-14.

### Example 4

### 1) Induction if Pf4 Toxin Gene

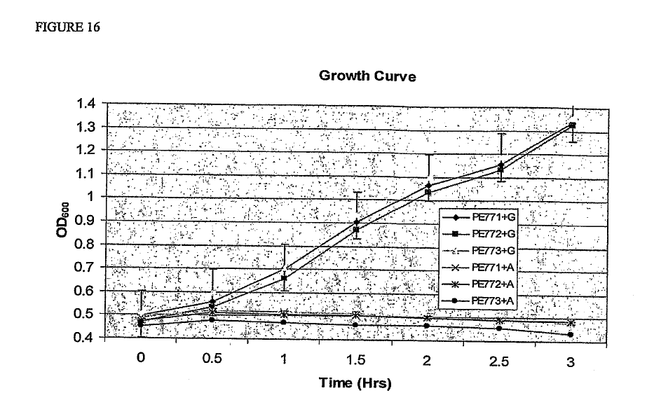

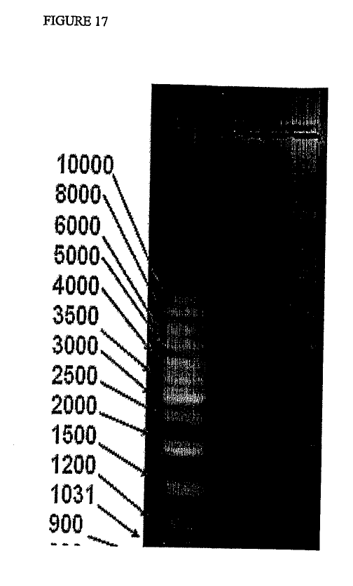

It can be seen from the FIG. 16 that induction with Arabinose on the PE771, PE772 and P773 constructs (Pf4 ParE-like toxin under control of Arabinose promoter). There is no significant increase in the OD₆₀₀ of Escherichia coli cultures for up to 3 hours. Slight dips in the OD from 2.5 hours can be observed.

This proves that the Pf4 toxin as expressed in PE771, 772 & 773 are bacteriostatic at the least.

Plate count data using LB ampicillin and either glucose to repress the toxin or arabinose to induce expression are shown below:

| | GLU 0.2% | ARA 1% |
|---|---|---|
| PE771 | >1 × 10⁹ | 1 × 10⁶ |
| PE772 | >1 × 10⁹ | <1 × 10⁴ |
| PE773 | >1 × 10⁹ | 1 × 10⁶ |

Briefly, overnight starter cultures were grown to OD600 of 0.5 before induction with 1% arabinose, before plating out on the respective LB ampicillin plates with arabinose (1%).

### 2) Insertional Inactivation

Using the EcoRI site existing within the Toxin gene a fragment of 1.8 kb was inserted into EcoRI digested PE773. The ligated mixture was then transformed back into E. coli for expression studies. Due to the insertional inactivation of the toxin gene it was found that colonies that grew on LB ampicillin with arabinose (1%) were those that carried the insert within the EcoRI site of the Toxin gene in PE773.

PCR results indicate that there are 2 types of inserts in this reaction. This colony carried 2 clones having a 2 kb insert and a 3.5 kb insert. The PCR reaction was done with primers for the cloning of the toxin gene. When purified further was found to be two separate clones.

### 3) Plasmid Stabilization Studies

The complete operon of the Pf4 antitoxin-toxin cassette including its putative regulatory sequences was cloned into pTRCHIS2 resulting in pTRC-PDPE-170 and transformed into TOP10 E. coli. The clones were then grown for 250 generations to the same cell density. The plasmid DNA were then extracted form the cultures for quantification. From the results it can be seen that the PDPE was able to stabilize the plasmid ensuring stable inheritance of the plasmid carrying the PDPE stabilization cassette.

Constructs: PCR cloned operon comprising Pf4 phage antitoxin-toxin gene under trc (IPTG induction) control. Cognate clone carries putative/proposed promoter/regulatory sequences for autoregulation.

Methods: Clones carrying 2 clones: C1 clone is the desired clone, C2 clone is the control where the clone is in reverse order—this will allow silencing using the IPTG promoter (this will be the experimental control).

At fixed OD600 of 1. The ratio of plasmid maintained in the culture is 1.8 folds higher than the silenced operon over 250 generations.

At least 4-fold stabilisation is possible. The data for the ratio is tabulated as follows:

| Induction | Silenced | Cognate | IPTG induced |
|---|---|---|---|
| Quantity ng/ml | 102.4 | 184.3 | 158.0 |
| Ratio over silenced | | 1.8 | 1.5 |

### REFERENCES

1. Agol, V. I. 1976. An aspect on the origin and evolution of viruses. Orig. Life. 7:119-132.
2. Ali, A., M. H. Rashid, and D. K. Karaolis. 2002. High-frequency rugose exopolysaccharide production by Vibrio cholerae. Appl. Environ. Microbiol. 68:5773-5778.
3. Bjedov, I., O. Tenaillon, B. Gerard, V. Souza, E. Denamur, M. Radman, F. Taddei, and I. Matic. 2003. Stress-induced mutagenesis in bacteria. Science 300:1404-1409.
4. Bradley, D. E. 1973. The adsorption of the Pseudomonas aeruginosa filamentous bacteriophage Pf to its host. Can. J. Microbiol. 19:623-631.
5. Brinton, C. C., Jr. 1971. The properties of sex pili, the viral nature of “conjugal” genetic transfer systems, and some possible approaches to the control of bacterial drug resistance. CRC Crit. Rev. Microbiol. 1:105-160.
6. Buell, C. R., V. Joardar, M. Lindeberg, J. Selengut, I. T. Paulsen, M. L. Gwinn, R. J. Dodson, R. T. Deboy, A. S. Durkin, J. F. Kolonay, R. Madupu, S. Daugherty, L. Brinkac, M. J. Beanan, D. H. Haft, W. C. Nelson, T. Davidsen, N. Zafar, L. Zhou, J. Liu, Q. Yuan, H. Khouri, N. Fedorova, B. Tran, D. Russell, K. Berry, T. Utterback, S. E. Van Aken, T. V. Feldblyum, M. D'Ascenzo, W. L. Deng, A. R. Ramos, J. R. Alfano, S. Cartinhour, A. K. Chatterjee, T. P. Delaney, S. G. Lazarowitz, G. B. Martin, D. J. Schneider, X. Tang, C. L. Bender, O. White, C. M. Fraser, and A. Collmer. 2003. The complete genome sequence of the Arabidopsis and tomato pathogen Pseudomonas syringae pv. tomato DC3000. Proc. Natl. Acad. Sci. USA. 100:10181-10186.
7. Busscher, H. J., and A. H. Weerkamp. 1987. Specific and non-specific interactions in bacterial adhesion to solid substrata. FEMS Microbiol. Rev. 46.
8. Canchaya, C., C. Proux, G. Fournous, A. Bruttin, and H. Brussow. 2003. Prophage genomics. Microbiol. Mol. Biol. Rev. 67:238-276.
9. Dasgupta, T., T. R. de Kievit, H. Masoud, E. Altman, J. C. Richards, I. Sadovskaya, D. P. Speert, and J. S. Lam. 1994. Characterization of lipopolysaccharide-deficient mutants of Pseudomonas aeruginosa derived from serotypes O3, O5, and O6. Infect. Immun. 62:809-817.
10. DeBeer, D., P. Stoodley, F. Roe, and Z. Lewandowski. 1994. Effects of biofilm structures on oxygen distribution and mass-transport. Biotech. Bioeng. 43:1131-1138.
11. Deretic, V., M. J. Schurr, J. C. Boucher, and D. W. Martin. 1994. Conversion of Pseudomonas aeruginosa to mucoidy in cystic fibrosis: environmental stress and regulation of bacterial virulence by alternative sigma factors. J. Bacteriol. 176:2773-2780.
12. Deziel, E., Y. Comeau, and R. Villemur. 2001. Initiation of biofilm formation by Pseudomonas aeruginosa 57RP correlates with emergence of hyperpiliated and highly adherent phenotypic variants deficient in swimming, swarming, and twitching motilities. J. Bacteriol. 183:1195-1204.
13. Dobrindt, U., and J. Reidl. 2000. Pathogenicity islands and phage conversion: evolutionary aspects of bacterial pathogenesis. Int. J. Med. Microbiol. 290:519-527.
14. Drenkard, E., and F. M. Ausubel. 2002. Pseudomonas biofilm formation and antibiotic resistance are linked to phenotypic variation. Nature 416:740-743.
15. Engelberg-Kulka, H., and G. Glaser. 1999. Addiction modules and programmed cell death and antideath in bacterial cultures. Annu. Rev. Microbiol. 53:43-70.
16. Ghigo, J. M. 2001. Natural conjugative plasmids induce bacterial biofilm development. Nature 412:442-445.
17. Hausner, M., and S. Wuertz. 1999. High rates of conjugation in bacterial biofilms as determined by quantitative in situ analysis. Appl. Environ. Microbiol. 65:3710-3713.
18. Häuβler, S., B. Tümmler, H. Weissbrodt, M. Rohde, and I. Steinmetz. 1999. Small-colony variants of Pseudomonas aeruginosa in cystic fibrosis. Clin. Infect. Dis. 29:621-625.
19. Häuβler, S., I. Ziegler, A. Löttel, F. von Götz, M. Rohde, D. Wehmhöhner, S. Saravanamuthu, B. Tümmler, and I. Steinmetz. 2003. Highly adherent small-colony variants of Pseudomonas aeruginosa in cystic fibrosis lung infection. J. Med. Microbiol. 52:295-301.
20. Hendrickx, L., M. Hausner, and S. Wuertz. 2003. Natural genetic transformation in monoculture Acinetobacter sp. strain BD413 biofilms. Appl. Environ. Microbiol. 69:1721-1727.
21. Hentzer, M., M. Givskov, and L. Eberl. 2004. Quorum Sensing in Biofilms: Gossip in Slime City. In M. Ghannoum and G. A. O'Toole (ed.), Microbial biofilms. ASM Press, Washington, D.C. pp. 478.
22. Heydorn, A., B. Ersboll, J. Kato, M. Hentzer, M. R. Parsek, T. Tolker-Nielsen, M. Givskov, and S. Molin. 2002. Statistical analysis of Pseudomonas aeruginosa biofilm development: impact of mutations in genes involved in twitching motility, cell-to-cell signaling, and stationary-phase sigma factor expression. Appl. Environ. Microbiol. 68:2008-2017.
23. Heydorn, A., A. T. Nielsen, M. Hentzer, C. Sternberg, M. Givskov, B. K. Ersboll, and S. Molin. 2000. Quantification of biofilm structures by the novel computer program COMSTAT. Microbiology 146:2395-2407.
24. Hill, D. F., N. J. Short, R. N. Perham, and G. B. Petersen. 1991. DNA sequence of the filamentous bacteriophage Pf1. J. Mol. Biol. 218:349-364.
25. Hohn, B., H. von Schutz, and D. A. Marvin. 1971. Filamentous bacterial viruses (II): killing of bacteria by abortive infection with fd. J. Mol. Biol. 56:155-165.
26. Holloway, B. W., V. Krishnapillai, and A. F. Morgan. 1979. Chromosomal genetics of Pseudomonas. Microbiol. Rev. 43:73-102.
27. Johnson, E. P., A. R. Strom, and D. R. Helinski. 1996. Plasmid RK2 toxin protein ParE: purification and interaction with the ParD antitoxin protein. J. Bacteriol. 178:1420-1429.
28. Kim, S. H., K. B. Lee, J. S. Lee, and Y. H. Cho. 2003. Genome diversification by phage-derived genomic islands in Pseudomonas aeruginosa. J. Microbiol. Biotechnol. 13:783-788.
29. Klausen, M., A. Heydorn, P. Ragas, L. Lambertsen, A. Aaes-Jorgensen, S. Molin, and T. Tolker-Nielsen. 2003. Biofilm formation by Pseudomonas aeruginosa wildtype, flagella, and type IV pili mutants. Mol. Microbiol. 48:1511-1524.
30. Kuo, M. Y., M. K. Yang, W. P. Chen, and T. T. Kuo. 2000. High-frequency interconversion of turbid and clear plaque strains of bacteriophage f1 and associated host cell death. Can. J. Microbiol. 46:841-847.
31. Kuo, T. T., C. C. Chiang, S. Y. Chen, J. H. Lin, and J. L. Kuo. 1994. A long lytic cycle in filamentous phage Cf1tv infecting Xanthomonas campestris pv. citri. Arch. Virol. 135:253-264.
32. Kutsukake, K., and T. Iino. 1980. Inversions of specific DNA segments in flagellar phase variation of Salmonella and inversion systems of bacteriophages P1 and Mu. Proc. Natl. Acad. Sci. USA 77:7338-7341.
33. Lehnherr, H., E. Maguin, S. Jafri, and M. B. Yarmolinsky. 1993. Plasmid addiction genes of bacteriophage P1: doc, which causes cell death on curing of prophage, and phd, which prevents host death when prophage is retained. J. Mol. Biol. 233:414-428.
34. Lin, S. H., W. P. Chen, and T. T. Kuo. 2001. Mechanism of host cell death induced by infection of Escherichia coli with the c2 clear-plaque mutant of phage f1. Botanical Bull. Acad. Sin. 42:45-52.
35. Liu, D. J., and L. A. Day. 1994. Pf1 virus structure: helical coat protein and DNA with paraxial phosphates. Science 265:671-674.
36. Marshall, K. C. 1985. Mechanisms of bacterial adhesion at solid-water interfaces. In D. C. Savage and M. Fletcher (ed.), Bacterial adhesion—mechanisms and physiological significance. Plenum Press, New York.
37. Martin, C., M. A. Ichou, P. Massicot, A. Goudeau, and R. Quentin. 1995. Genetic diversity of Pseudomonas aeruginosa strains isolated from patients with cystic fibrosis revealed by restriction fragment length polymorphism of the rRNA gene region. J. Clin. Microbiol. 33:1461-1466.
38. Marvin, D. A. 1998. Filamentous phage structure, infection and assembly. Current Opinion in Structural Biology 8:150-158.
39. Matsukawa, M., and E. P. Greenberg. 2004. Putative exopolysaccharide synthesis genes influence Pseudomonas aeruginosa biofilm development. J Bacteriol 186:4449-4456.
40. Miller, R. V. 2001. Environmental bacteriophage-host interactions: factors contribution to natural transduction. Antonie Van Leeuwenhoek 79:141-147.
41. Molin, S., and T. Tolker-Nielsen. 2003. Gene transfer occurs with enhanced efficiency in biofilms and induces enhanced stabilisation of the biofilm structure. Curr. Opin. Biotechnol. 14:255-261.
42. Moller, S., C. Sternberg, J. B. Andersen, B. B. Christensen, J. L. Ramos, M. Givskov, and S. Molin. 1998. In situ gene expression in mixed-culture biofilms: evidence of metabolic interactions between community members. Appl. Environ. Microbiol. 64:721-732.
43. Oliver, A., R. Canton, P. Campo, F. Baquero, and J. Blazquez. 2000. High frequency of hypermutable Pseudomonas aeruginosa in cystic fibrosis lung infection. Science 288:1251-1254.
44. O'Toole, G. A., and R. Kolter. 1998. Flagellar and twitching motility are necessary for Pseudomonas aeruginosa biofilm development. Mol. Microbiol. 30:295-304.
45. O'Toole, G. A., and R. Kolter. 1998. Initiation of biofilm formation in Pseudomonas fluorescens WCS365 proceeds via multiple, convergent signalling pathways: a genetic analysis. Mol. Microbiol. 28:449-461.
46. Pratt, D., H. Tzagoloff, and W. S. Erdahl. 1966. Conditional lethal mutants of the small filamentous coliphage (I): isolation, complementation, cell killing, time of cistron action. Virol. 30:397-410.
47. Reisner, A., J. A. Haagensen, M. A. Schembri, E. L. Zechner, and S. Molin. 2003. Development and maturation of Escherichia coli K-12 biofilms. Mol. Microbiol. 48:933-946.
48. Sadowska, B., A. Bonar, C. von Eiff, R. A. Proctor, M. Chmiela, W. Rudnicka, and B. Rozalska. 2002. Characteristics of Staphylococcus aureus, isolated from airways of cystic fibrosis patients, and their small colony variants. FEMS Immunol. Med. Microbiol. 32:191-197.
49. Saner, K., A. K. Camper, G. D. Ehrlich, J. W. Costerton, and D. G. Davies. 2002. Pseudomonas aeruginosa displays multiple phenotypes during development as a biofilm. J. Bacteriol. 184:1140-1154.
50. Sauvonnet, N., P. Gounon, and A. P. Pugsley. 2000. PpdD Type IV Pilin of Escherichia coli K-12 Can Be Assembled into Pili in Pseudomonas aeruginosa. J. Bacteriol. 182:848-854.
51. Shieh, G. J., Y. C. Charng, B. C. Yang, T. Jenn, H. J. Bau, and T. T. Kuo. 1991. Identification and nucleotide sequence analysis of an open reading frame involved in high-frequency conversion of turbid to clear plaque mutants of filamentous phage Cf1t. Virol. 185:316-322.
52. Stanley, N. R., R. A. Britton, A. D. Grossman, and B. A. Lazazzera. 2003. Identification of catabolite repression as a physiological regulator of biofilm formation by Bacillus subtilis by use of DNA microarrays. J. Bacteriol. 185:1951-1957.
53. Sternberg, C., B. B. Christensen, T. Johansen, A. Toftgaard Nielsen, J. B. Andersen, M. Givskov, and S. Molin. 1999. Distribution of bacterial growth activity in flow-chamber biofilms. Appl. Environ. Microbiol. 65:4108-4117.
54. Stover, C. K., X. Q. Pham, A. L. Erwin, S. D. Mizoguchi, P. Warrener, M. J. Hickey, F. S. Brinkman, W. O. Hufnagle, D. J. Kowalik, M. Lagrou, R. L. Garber, L. Goltry, E. Tolentino, S. Westbrock-Wadman, Y. Yuan, L. L. Brody, S. N. Coulter, K. R. Folger, A. Kas, K. Larbig, R. Lim, K. Smith, D. Spencer, G. K. Wong, Z. Wu, L. T. Paulsen, J. Reizer, M. H. Saier, R. E. Hancock, S. Lory, and M. V. Olson. 2000. Complete genome sequence of Pseudomonas aeruginosa PA01, an opportunistic pathogen. Nature 406:959-964.
55. Taddei, F., I. Matic, and M. Radman. 1995. cAMP-dependent SOS induction and mutagenesis in resting bacterial populations. Proc. Natl. Acad. Sci. USA 92:11736-11740.
56. Tolker-Nielsen, T., U. C. Brinch, P. C. Ragas, J. B. Andersen, C. S. Jacobsen, and S. Molin. 2000. Development and dynamics of Pseudomonas sp. biofilms. J. Bacteriol. 182:6482-6489.
57. Tominaga, A. 1997. The site-specific recombinase encoded by pinD in Shigella dysenteriae is due to the presence of a defective Mu prophage. Microbiol. 143:2057-2063.
58. Wagner, V. E., D. Bushnell, L. Passador, A. L. Brooks, and B. H. Iglewski. 2003. Microarray analysis of Pseudomonas aeruginosa quorum-sensing regulons: Effects of growth phase and environment. Journal of Bacteriology 185:2080-2095.
59. Webb, J. S., L. S. Thompson, S. James, T. Charlton, T. Tolker-Nielsen, B. Koch, M. Givskov, and S. Kjelleberg. 2003. Cell death in Pseudomonas aeruginosa biofilm development. J. Bacteriol. 185:4585-4592.
60. Welsh, L. C., M. F. Symmons, and D. A. Marvin. 2000. The molecular structure and structural transition of the alpha-helical capsid in filamentous bacteriophage Pf1. Acta Crystallogr. D. Biol. Crystallogr. 56:137-150.
61. Whitchurch, C. B., and J. S. Mattick. 1994. Characterization of a gene, pilU, required for twitching motility but not phage sensitivity in Pseudomonas aeruginosa. Mol. Microbiol. 13:1079-1091.
62. Whitchurch, C. B., T. Tolker-Nielsen, P. C. Ragas, and J. S. Mattick. 2002. Extracellular DNA required for bacterial biofilm formation. Science 295:1487.
63. Whiteley, M., M. G. Bangera, R. E. Bumgarner, M. R. Parsek, G. M. Teitzel, S. Lory, and E. P. Greenberg. 2001. Gene expression in Pseudomonas aeruginosa biofilms. Nature 413:860-864.

1. A Phd-like (prevent host death) antitoxin protein which protein comprises the sequence as set forth in SEQ ID NO: 1 or which comprises a functional equivalent thereof.

2. A ParE-like toxin protein which protein: (i) comprises the sequence as set forth in SEQ ID NO: 2 or SEQ ID NO: 5; or (ii) which comprises a functional equivalent of (i).

3. The protein according to claim 1 wherein the protein consists of the sequence as set forth in SEQ ID NO:1.

4. The protein according to claim 2 wherein the protein consists of the sequence as set forth in SEQ ID NO: 2 or SEQ ID NO: 5.

5. The protein according to claim 1 wherein the protein comprises a functional equivalent of the protein set forth in SEQ ID NO: 1 and wherein the functional equivalent is at least about 80, 85, 90, 92, 94, 95, 96, 97, 98, 99 or 99.5 percent sequentially identical over its entire length as compared to SEQ ID NO: 1.

6. The protein according to claim 2 wherein the protein comprises a functional equivalent of the protein set forth in SEQ ID NO: 2 and wherein the functional equivalent is at least about 80, 85, 90, 92, 94, 95, %, 97, 98, 99 or 99.5 percent sequentially identical over its entire length as compared with SEQ ID NO: 2 or SEQ ID NO: 5.

7. The protein according to claims 1 or 2 which is provided in the form of a fusion protein or which is a fragment which retains biological or immunological activity.

8. A nucleic acid molecule which encodes an antitoxin protein according to claims 1 or 2.

9. (canceled)

10. A plasmid which comprises a nucleic acid sequence of interest and which replicates in bacteria and which is stabilized by the presence of a nucleic acid sequence which encodes an antitoxin protein according to claim 1 and a nucleic acid sequence which encodes a toxin protein according to claim 2.

11. A plasmid which comprises a nucleic acid sequence of interest and which replicates in bacteria and which comprises a nucleic acid sequence which encodes an antitoxin protein according to claim 1.

12. A plasmid which comprises a nucleic add sequence which encodes a toxin protein according to claim 2, whereby the expression of the parE-like toxin protein is driven by a constitutive or selectable expression promoter and wherein said nucleic acid sequence comprises a multiple cloning site (MCS) to thereby facilitate the insertion of a nucleic acid sequence of interest.

13. The plasmid according to claim 12 wherein a nucleic acid sequence of interest has been inserted into the MCS of the plasmid.

14. The plasmid according to claim 12 wherein the plasmid further comprises a selectable marker.

15. A bacterium transformed with a plasmid according to anyone of claims 10 to 12.

16. The bacterium according to claim 15 wherein the bacterium comprises a plasmid according to claim 11 and wherein the bacterial chromosome has been irreversibly altered so as to produce a protein according to claim 2 which is toxic to the bacterium.

17. A method to replicate DNA contained in a plasmid according to anyone of claims 10 to 12 which method comprises culturing bacterial cells according to claim 15.

18. A method of producing a protein of interest, the method comprising
culturing, bacterial cells of according to claim 15 under conditions whereby said protein of interest is expressed from the nucleic acid sequence of interest and
recovering said protein of interest thus produced.

19. (canceled)

20. (canceled)

21. A pharmaceutical composition comprising the bacterium according claim to 15.

22. A method for vaccinating a subject comprising administering to the subject a pharmaceutical composition according to claim 21.

23. (canceled)

24. (canceled)

25. A method for identifying an agonist or antagonist compound of a protein according to any one of claims 1 to 2, the method comprising the steps of
contacting a test compound with the protein according to claim 1 or 2 and
determining if the test compound binds to the protein.

26. (canceled)

27. The method according to claim 25 wherein the method further comprises determining if the test compound enhances or decreases the activity of the protein.

28. A method for identifying a compound that is effective to alter the expression of a target polynucleotide which encodes a polypeptide according to claim 1 or 2, the method comprising
a) exposing a sample comprising the target polynucleotide to a test compound,
b) detecting altered expression, if any, of the target polynucleotide.

29. A method of modulating bacterial cell growth, the method comprising contacting the cells whose growth is to be controlled with a protein according to claim 1 or 2.

30. The method according to claim 29 wherein the method is a method of treating or preventing a bacterial infection in a patient in need thereof comprising administering to the patient a protein according claim 2.

31. (canceled)

32. (canceled)

33. A pharmaceutical composition comprising a protein according to claim 1 or 2.

34. The method according to claim 30 wherein the patient is a cystic fibrosis patient.

35. The method according to claim 29 wherein the method is a method of cleaning, disinfecting, or decontaminating a surface, the method comprising contacting the surface with a cleaning composition comprising a protein according to claim 2.

36. A kit comprising a protein according to claim 2 and an agent, for dismantling a biofilm, wherein said agent is an enzyme.
